(19) United States
(12) Patent Application Publication
(10) Pub. No.: US 2019/0284536 A1
(43) Pub. Date: Sep. 19, 2019

(54) EPITHELIAL TUMOR CELL CULTURES

(71) Applicant: Beijing Percans Oncology Co., Ltd., Beijing (CN)

(72) Inventors: Yihong ZHANG, Calabasas, CA (US); Yang LIU, Beijing (CN); Zhen YANG, Beijing (CN)

(21) Appl. No.: 16/348,705

(22) PCT Filed: Nov. 21, 2017
(86) PCT No.: PCT/US17/62863

§ 371 Date: May 9, 2019

(30) Foreign Application Priority Data
Nov. 21, 2016 (CN) PCT/CN2016/106619
May 31, 2017 (CN) PCT/CN2017/086556

(51) Int. Cl. C12N 5/09; C12N 5/00; C07K 14/76; C12N 5/077

(52) U.S. Cl. CPC C12N 5/0693; C12N 5/0018; C12N 2501/734; C12N 5/0656; C07K 14/76

(57) ABSTRACT

Provided are cell cultures and related methods for the enrichment and conditional reprogramming of patient-derived, primary epithelial tumor cells from cancer-tissue originated spheroids (CTOSs). Also provided are methods of evaluating the responsiveness of the epithelial tumor cells to one or more therapeutic agents.

### CROSS-REFERENCE TO RELATED APPLICATIONS

This application claims priority to PCT/CN2017/086556, filed 31 May 2017; and PCT/CN2016/106619, filed 21 Nov. 2016, each of which is incorporated by reference in its entirety.

### BACKGROUND

### Technical Field

Embodiments of the present disclosure relate to cell cultures and related methods for the enrichment and conditional reprogramming of patient-derived, primary epithelial tumor cells from cancer-tissue originated spheroids (CTOSs). Also included are methods of evaluating the responsiveness of the epithelial tumor cells to one or more therapeutic agents.

### Description of the Related Art

Despite constant efforts to improve diagnostics and therapeutics, cancer remains a leading cause of death worldwide. The diversity or heterogeneity of cancer presents an obstacle to the development of new therapies and also makes it difficult to identify likely responders. Moreover, many cancer therapies are challenged by primary and acquired resistance, including additional point mutations and alternative pathways that bypass the targets of therapeutic reagents.

Future successful therapies will likely rely on a comprehensive analysis of events underlying the tumor progression and the metastatic processes, together with the development of a rapid and non-selective generation of patient-derived primary tumor cells that could be easily manipulated in order to accurately evaluate the efficacy of chemotherapeutic agents and targeted therapeutic agents.

As one example, a primary culture of cancer cells using irradiated feeder cells and Rho kinase inhibitor Y27632 has been describe to promote the growth of the heterogenous epithelial tumor cells and to investigate drug sensitivity for individual patients (see, for example, Liu et al., Am J Pathol. 180:599-607, 2012). However, these co-culture conditions are plagued by the overgrowth of stromal cells when the isolated single cells from patient tumors are merely seeded.

One approach to solve this stromal cell contamination is to use FACS to purify the epithelial cell adhesion molecule (EpCAM) positive cell subpopulation. But purifications based upon cell surface marker expression increase the risk of clonal selection of the tumor cells derived from original patient tumor, and thus often fail to fairly represent the clonal diversity of the original patient tumor.

Thus, there is a need to develop improved culture conditions for enriching primary epithelial tumor cells in a feeder cell and Rock inhibitor system, which not only reduce stromal cell growth but also maintain the clonal diversity of the original tumor sample.

### BRIEF SUMMARY

Embodiments of the present disclosure include a cell culture medium, comprising

(a) human ex vivo-derived cancer-tissue originated spheroids (CTOSs) which are dissociated into a substantially single cell suspension, wherein the CTOSs comprise human epithelial tumor cells,

(b) feeder cells; and

(c) a defined cell culture medium that comprises at least one Rho-associated, coiled-coil containing protein kinase (ROCK) inhibitor; and optionally

(d) supplemental serum albumin (SA),

wherein the cell culture medium provides at least about 10% proliferation of the epithelial tumor cells within about 14 days following co-culture of the dissociated CTOSs with (b) and (c).

In some instances, the defined cell culture medium does not comprise or is substantially free of a

Bone Morphogenetic Protein (BMP) inhibitor such as Noggin and a Wnt/β-catenin signaling agonist such as R-spondin-1.

In certain embodiments, the human ex vivo-derived CTOSs are cultured from a tumor-containing sample removed from a human patient with cancer, for example, selected from a surgical sample, a biopsy sample, a pleural effusion sample, and an ascetic fluid sample. In certain embodiments, the human patient has a cancer selected from colon cancer, lung cancer, gastric cancer, and breast cancer.

In certain embodiments, the epithelial tumor cells substantially retain the clonal diversity of the tumor-containing sample removed from the human patient within about 14 days following co-culture of the dissociated CTOSs with (b) and (c).

In some embodiments, the human epithelial tumor cells are selected from colon cancer cells, lung cancer cells, gastric cancer cell, and breast cancer cells.

In some embodiments, the cell culture medium provides at least about 10, 20, 30, 40, 50, 60, 70, 80, 90, or 100% proliferation of the human epithelial tumor cells within about 14, 13, 12, 11, 10, 9, 8, 7, 6, 5, 4, 3, 2 or 1 days following co-culture of the dissociated CTOSs with (b) and (c). In certain embodiments, the cell culture medium provides at least about 20-30% proliferation of the human epithelial tumor cells within about 7 days following co-culture of the dissociated CTOSs with (b) and (c). In certain embodiments, the cell culture medium provides at least about 60% proliferation of the human epithelial tumor cells within about 7 days following co-culture of the dissociated CTOSs with (b) and (c).

In certain embodiments, the cell culture medium provides a ratio of about or at least about 5:1, 10:1, 20:1, 50:1, 100:1, 200:1, 500:1, or 1000:1 between the human epithelial tumor cells and human stromal cells within about 14 days following co-culture of the dissociated CTOSs with (b) and (c), wherein the human epithelial tumor cells are characterized, for example, by cell surface expression of epithelial cell adhesion molecule (EpCAM) and/or CD133.

In some embodiments, the defined medium comprises serum, for example, fetal bovine serum (FBS). In some embodiments, the concentration of the serum ranges from about 1-5%, or is about 1%, 2%, 3%, 4%, or 5%.

In some embodiments, the ROCK inhibitor is selected from one or more of Y27632, HA1100 hydrochloride, HA1077, and GSK429286. In some embodiments, the concentration of Y27632 ranges from about 0.1-1000, 0.5-1000, 1-1000, 5-1000, 10-1000, 50-1000, 100-1000, 500-1000μM, or from about 0.1-500, 0.5-500, 1-500, 5-500, 10-500, 50-500, 100-500 μM, or from about 0.1-100, 0.5-100, 1-100, 5-100, 10-100, 50-100 μM, or from about 0.1-50, 0.5-50, 1-50, 5-50, 10-50 μM, or from about 0.1-40, 0.5-40, 1-40, 5-40, 10-40 μM, or from about 0.1-30, 0.5-30, 1-30, 5-30, 10-30 μM, or from about 0.1-20, 0.5-20, 1-20, 5-20, 10-20 μM, or from about 0.1-10, 0.5-10, 1-10, 5-10 μM, or from about 0.1-5, 0.5-5, 1-5 μM, or from about 0.1-1 or 0.5-1 μM.

In some embodiments, the supplemental SA is selected from bovine serum albumin (BSA) and human serum albumin (HSA). In certain embodiments, the concentration of the supplemental SA is from about 0.5 mg/ml to about 20 mg/ml, or about, less than about, or no more than about 0.5, 0.6, 0.7, 0.8, 0.9, 1.0, 1.2, 1.4, 1.6, 1.8, 2.0, 2.2, 2.5, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, 10.0, 10.2, 10.4, 10.6, 10.8, 11.0, 11.2, 11.4, 11.6, 11.8, 12.0, 12.2, 12.4, 12.6, 12.8, 13.0, 13.2, 13.4, 13.6, 13.8, 14.0, 14.2, 14.4, 14.6, 14.8, 15.0, 15.2, 15.4, 15.6, 15.8, 16.0, 16.2, 16.4, 16.6, 16.8, 17.0, 17.2, 17.4, 17.6, 17.8, 18.0, 18.2, 18.4, 18.6, 18.8, 19.0, 19.2, 19.4, 19.6, 19.8, or 20 mg/ml.

In some embodiments, the feeder cells are non-proliferating fibroblasts, for example, human fibroblasts.

Also included are tissue culture plates having a low cell binding surface, comprising a cell culture medium described herein.

Some embodiments include methods of culturing or expanding human epithelial tumor cells in an in vitro cell culture medium, comprising

(A) dissociating human ex vivo-derived cancer-tissue originated spheroids (CTOSs) which comprise the human epithelial tumor cells into a substantially single cell suspension; and

(B) co-culturing the dissociated CTOSs with feeder cells in a defined cell culture medium that comprises at least one Rho-associated, coiled-coil containing protein kinase (ROCK) inhibitor,

wherein the method provides at least about 10% proliferation of the epithelial tumor cells in the cell culture medium within about 14 days following steps (A) and (B).

In some instances, the defined cell culture medium does not comprise or is substantially free of a Bone Morphogenetic Protein (BMP) inhibitor such as Noggin and a Wnt/β-catenin signaling agonist such as R-spondin-1. In some embodiments, the cell culture medium comprises supplemental serum albumin (SA).

In some embodiments, the substantially dissociated CTOSs are co-cultured in a tissue culture plate having a low cell binding surface.

In some embodiments, the human ex vivo-derived CTOSs are cultured from a tumor sample removed from a human patient with cancer, for example, which is selected from a surgical sample, a biopsy sample, a pleural effusion sample, and an ascetic fluid sample. In some embodiments, the human patient has a cancer selected from colon cancer, lung cancer, gastric cancer, and breast cancer. In some embodiments, the epithelial tumor cells substantially retain the clonal diversity of the tumor sample removed from the human patient within about 14 days following steps (A) and (B).

In some embodiments, the human epithelial tumor cells are selected from colon cancer cells, lung cancer cells, gastric cancer cell, and breast cancer cells.

In some embodiments, the method or cell culture medium provides at least about 10, 20, 30, 40, 50, 60, 70, 80, 90, or 100% proliferation of the human epithelial tumor cells in the cell culture medium within about 14, 13, 12, 11, 10, 9, 8, 7, 6, or 5 days following steps (A) and (B). In some embodiments, the method or cell culture medium provides at least about 20-30% proliferation of the human epithelial tumor cells in the cell culture medium within about 7 days following steps (A) and (B). In some embodiments, the method or cell culture medium provides at least about 60% proliferation of the human epithelial tumor cells in the cell culture medium within about 7 days following steps (A) and (B).

In particular embodiments, the method or cell culture medium provides a ratio of at least about 5:1, 10:1, 20:1, 50:1, 100:1, 200:1, 500:1, or 1000:1 between the human epithelial tumor cells and human stromal cells in the cell culture medium within about 14 days following steps (A) and (B), wherein the human epithelial tumor cells are characterized, for example, by cell surface expression of epithelial cell adhesion molecule (EpCAM) and/or CD133.

In some embodiments, the defined cell culture medium comprises serum, optionally fetal bovine serum (FBS). In some embodiments, the concentration of the serum ranges from about 1-5%, or is about 1%, 2%, 3%, 4%, or 5%.

In some embodiments, the ROCK inhibitor is selected from one or more of Y27632, HA1100 hydrochloride, HA1077, and GSK429286. In some embodiments, the concentration of the ROCK inhibitor, for example, Y27632, ranges from about 0.1-1000, 0.5-1000, 1-1000, 5-1000, 10-1000, 50-1000, 100-1000, 500-1000 μM, or from about 0.1-500, 0.5-500, 1-500, 5-500, 10-500, 50-500, 100-500 μM, or from about 0.1-100, 0.5-100, 1-100, 5-100, 10-100, 50-100 μM, or from about 0.1-50, 0.5-50, 1-50, 5-50, 10-50 μM, or from about 0.1-40, 0.5-40, 1-40, 5-40, 10-40 μM, or from about 0.1-30, 0.5-30, 1-30, 5-30, 10-30 μM, or from about 0.1-20, 0.5-20, 1-20, 5-20, 10-20 μM, or from about 0.1-10, 0.5-10, 1-10, 5-10 μM, or from about 0.1-5, 0.5-5, 1-5 μM, or from about 0.1-1 or 0.5-1 μM.

In some embodiments, the supplemental SA is selected from bovine serum albumin (BSA) and human serum albumin (HSA). In certain embodiments, the concentration of the supplemental SA is from about 0.5 mg/ml to about 20 mg/ml, or about, less than about, or no more than about 0.5, 0.6, 0.7, 0.8, 0.9, 1.0, 1.2, 1.4, 1.6, 1.8, 2.0, 2.2, 2.5, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, 10.0, 10.2, 10.4, 10.6, 10.8, 11.0, 11.2, 11.4, 11.6, 11.8, 12.0, 12.2, 12.4, 12.6, 12.8, 13.0, 13.2, 13.4, 13.6, 13.8, 14.0, 14.2, 14.4, 14.6, 14.8, 15.0, 15.2, 15.4, 15.6, 15.8, 16.0, 16.2, 16.4, 16.6, 16.8, 17.0, 17.2, 17.4, 17.6, 17.8, 18.0, 18.2, 18.4, 18.6, 18.8, 19.0, 19.2, 19.4, 19.6, 19.8, or 20 mg/ml.

In some embodiments, the feeder cells are non-proliferating fibroblasts, for example, human fibroblasts.

In some embodiments, the human ex vivo-derived CTOSs are cultured from a tumor-containing sample removed from a human patient with cancer, which comprises, for example,

mincing and incubating the tumor-containing sample in a tissue digestion medium, and

culturing the tumor-containing sample in a defined, serum-free, and feeder-free CTOS growth medium that comprises nicotinamide, Wnt3A, a Bone Morphogenetic Protein (BMP) inhibitor, a Wnt/β-catenin signaling agonist, and a ROCK inhibitor, for a time sufficient to form the CTOSs.

In some embodiments, the tissue digestion medium comprises a protease selected from one or more of collagenase I, collagenase II, a neutral non-clostridial protease, and any combination thereof.

In some embodiments, the concentration of nicotinamide ranges from about 0.1-1000, 0.5-1000, 1-1000, 5-1000, 10-1000, 50-1000, 100-1000, 500-1000 mM, or from about 0.1-500, 0.5-500, 1-500, 5-500, 10-500, 50-500, 100-500 mM, or from about 0.1-100, 0.5-100, 1-100, 5-100, 10-100, 50-100 mM, or from about 0.1-50, 0.5-50, 1-50, 5-50, 10-50 mM, or from about 0.1-40, 0.5-40, 1-40, 5-40, 10-40 mM, or from about 0.1-30, 0.5-30, 1-30, 5-30, 10-30 mM, or from about 0.1-20, 0.5-20, 1-20, 5-20, 10-20 mM, or from about 0.1-10, 0.5-10, 1-10, 5-10 mM, or from about 0.1-5, 0.5-5, 1-5 mM, or from about 0.1-1 or 0.5-1 mM.

In some embodiments, the concentration of Wnt3A ranges from about 0.1-10,000 or 1-1000 ng/ml, or from about 0.1-1000, 1-1000, 10-1000, 20-1000, 30-1000, 40-1000, 50-1000, 60-1000, 70-1000, 80-1000, 90-1000, 100-1000, 200-1000, 300-1000, 400-1000, 500-1000, 600-1000, 700-1000, 800-1000, 900-1000 ng/ml, or from about 0.1-500, 1-500, 10-500, 20-500, 30-500, 40-500, 50-500, 60-500, 70-500, 80-500, 90-500, 100-500, 200-500, 300-500, 400-500 ng/ml, or from about 0.1-400, 1-400, 10-400, 20-400, 30-400, 50-400, 40-400, 60-400, 70-400, 80-400, 90-400, 100-400, 200-400, 300-400 ng/ml, or from about 0.1-300, 1-300, 10-300, 20-300, 30-300, 40-300, 50-300, 60-300, 70-300, 80-300, 90-300, 100-300, 200-300 ng/ml, or from about 0.1-200, 1-200, 10-200, 20-200, 30-200, 40-200, 50-200, 60-200, 70-200, 80-200, 90-200, 100-200 ng/ml, or from about 0.1-150, 1-150, 10-150, 20-150, 30-150, 40-150, 50-150, 60-150, 70-150, 80-150, 90-150, 100-150 ng/ml, or from about 0.1-120, 1-120, 10-120, 20-120, 30-120, 40-120, 50-120, 60-120, 70-120, 80-120, 90-120, 100-120 ng/ml, or from about 0.1-100, 1-100, 10-100, 20-100, 30-100, 40-100, 50-100, 60-100, 70-100, 80-100, 90-100 ng/ml.

In some embodiments, the BMP inhibitor is noggin, and in some embodiments the concentration of the BMP inhibitor ranges from about 0.1-10,000 or 1-1000 ng/ml, or from about 0.1-1000, 1-1000, 10-1000, 20-1000, 30-1000, 40-1000, 50-1000, 60-1000, 70-1000, 80-1000, 90-1000, 100-1000, 200-1000, 300-1000, 400-1000, 500-1000, 600-1000, 700-1000, 800-1000, 900-1000 ng/ml, or from about 0.1-500, 1-500, 10-500, 20-500, 30-500, 40-500, 50-500, 60-500, 70-500, 80-500, 90-500, 100-500, 200-500, 300-500, 400-500 ng/ml, or from about 0.1-400, 1-400, 10-400, 20-400, 30-400, 50-400, 40-400, 60-400, 70-400, 80-400, 90-400, 100-400, 200-400, 300-400 ng/ml, or from about 0.1-300, 1-300, 10-300, 20-300, 30-300, 40-300, 50-300, 60-300, 70-300, 80-300, 90-300, 100-300, 200-300 ng/ml, or from about 0.1-200, 1-200, 10-200, 20-200, 30-200, 40-200, 50-200, 60-200, 70-200, 80-200, 90-200, 100-200 ng/ml, or from about 0.1-150, 1-150, 10-150, 20-150, 30-150, 40-150, 50-150, 60-150, 70-150, 80-150, 90-150, 100-150 ng/ml, or from about 0.1-120, 1-120, 10-120, 20-120, 30-120, 40-120, 50-120, 60-120, 70-120, 80-120, 90-120, 100-120 ng/ml, or from about 0.1-100, 1-100, 10-100, 20-100, 30-100, 40-100, 50-100, 60-100, 70-100, 80-100, 90-100 ng/ml.

In some embodiments, the Wnt/β-catenin signaling agonist is R-spondin-1, and in some embodiments the concentration of Wnt/β-catenin signaling agonist ranges from about 0.1-10,000 or 1-1000 or 100-1000 ng/ml, or from about 0.1-10,000, 1-10,000, 10-10,000, 20-10,000, 30-10,000, 40-10,000, 50-10,000, 60-10,000, 70-10,000, 80-10,000, 90-10,000, 100-10,000, 200-10,000, 300-10,000, 400-10,000, 500-10,000, 1000-10,000, 5000-10,000 ng/ml, or from about 0.1-5000, 1-5000, 10-5000, 20-5000, 30-10,000, 40-5000, 50-5000, 60-5000, 70-5000, 80-5000, 90-5000, 100-5000, 200-5000, 300-5000, 400-5000, 500-5000, 1000-5000 ng/ml, or from about 0.1-1000, 1-1000, 10-1000, 20-1000, 30-1000, 40-1000, 50-1000, 60-1000, 70-1000, 80-1000, 90-1000, 100-1000, 200-1000, 300-1000, 400-1000, 500-1000, 600-1000, 700-1000, 800-1000, 900-1000 ng/ml, or from about 0.1-800, 1-800, 10-800, 20-800, 30-800, 40-800, 50-800, 60-800, 70-800, 80-800, 90-800, 100-800, 200-800, 300-800, 400-800, 500-800, 600-800, 700-800 ng/ml, or from about 0.1-700, 1-700, 10-700, 20-700, 30-700, 40-700, 50-700, 60-700, 70-700, 80-700, 90-700, 100-700, 200-700, 300-700, 400-700, 500-700, 600-700 ng/ml, or from about 0.1-600, 1-600, 10-600, 20-600, 30-600, 40-600, 50-600, 60-600, 70-600, 80-600, 90-600, 100-600, 200-600, 300-600, 400-600, 500-600 ng/ml, or from about 0.1-500, 1-500, 10-500, 20-500, 30-500, 40-500, 50-500, 60-500, 70-500, 80-500, 90-500, 100-500, 200-500, 300-500, 400-500 ng/ml.

In some embodiments, the ROCK inhibitor is Y27632, and in some embodiments the concentration of the ROCK inhibitor ranges from about 0.1-1000, 0.5-1000, 1-1000, 5-1000, 10-1000, 50-1000, 100-1000, 500-1000 μM, or from about 0.1-500, 0.5-500, 1-500, 5-500, 10-500, 50-500, 100-500 μM, or from about 0.1-100, 0.5-100, 1-100, 5-100, 10-100, 50-100 μM or from about 0.1-50, 0.5-50, 1-50, 5-50, 10-50 μM, or from about 0.1-40, 0.5-40, 1-40, 5-40, 10-40 μM, or from about 0.1-30, 0.5-30, 1-30, 5-30, 10-30 μM, or from about 0.1-20, 0.5-20, 1-20, 5-20, 10-20 μM, or from about 0.1-10, 0.5-10, 1-10, 5-10 μM, or from about 0.1-5, 0.5-5, 1-5 μM, or from about 0.1-1 or 0.5-1 μM.

Also included are methods of testing responsiveness of a human patient to a therapeutic agent, comprising

administering the therapeutic agent to a cell culture medium or co-culture described herein, or to a cell culture medium or co-culture prepared by a method described herein; and

measuring epithelial tumor cell proliferation and/or epithelial tumor cell apoptosis,

wherein a decrease in epithelial tumor cell proliferation and/or an induction in epithelial tumor cell apoptosis is indicative of responsiveness of the human patient to the therapeutic agent, and wherein a lack of decrease in epithelial tumor cell proliferation and/or induction of epithelial tumor cell apoptosis is indicative of resistance of the human patient to the therapeutic agent.

Certain methods comprise administering the therapeutic agent on the same day as co-culturing the dissociated CTOSs. Certain methods comprise administering the therapeutic agent at least one day following co-culture of the dissociated CTOSs. Certain methods comprise administering the therapeutic agent about or within about 1, 2, 3, 4, 5, 6, or 7 days following co-culture of the dissociated CTOSs. Some methods comprise measuring epithelial tumor cell proliferation and/or epithelial tumor cell apoptosis within about 14 days of administering the therapeutic agent. Particular methods comprise measuring epithelial tumor cell proliferation and/or epithelial tumor cell apoptosis within about 14, 13, 12, 11, 10, 9, 8, 7, 6, or 5 days of administering the therapeutic agent. Certain methods comprise measuring epithelial tumor cell proliferation and/or epithelial tumor cell apoptosis within about 7, 6, or 5 days of administering the therapeutic agent.

In some embodiments, the step of measuring epithelial tumor cell proliferation comprises measuring a cellular proliferation marker. In some embodiments, the cellular proliferation marker is selected from one or more of ³H-thymidine, bromodeoxyuridine (BrdU), 5-ethynyl-2′-deoxyuridine (Edu), Ki-67, and proliferating cell nuclear antigen (PCNA).

In some embodiments, the step of measuring epithelial tumor cell apoptosis comprises measuring a cellular apoptosis marker. In some embodiments, the cellular apoptosis marker is selected from one or more of fluorochrome-labeled inhibitors of Caspases (FLICA), caspase activation, poly ADP ribose polymerase (PARP) cleavage, DRAQ5, DRAQ7, and terminal deoxynucleotidyl transferase (TdT) dUTP nick-end labeling (TUNEL) assay.

Also included are sample preparation kits, comprising any combination of:
a defined, serum-free, and feeder-free cancer-tissue originated spheroid (CTOSs) growth medium;
a tissue digestion medium;
a tissue culture plate having a low cell binding surface;
a defined cell culture medium;
frozen feeder cells;
a Rho-associated, coiled-coil containing protein kinase (ROCK) inhibitor; and optionally
a cellular proliferation marker and/or a cellular apoptosis marker.

In some kits, the CTOS growth medium comprises nicotinamide, Wnt3A, a Bone Morphogenetic Protein (BMP) inhibitor, a Wnt/β-catenin signaling agonist, and/or a ROCK inhibitor.

In some kits, the tissue digestion medium comprises a protease selected from one or more of collagenase I, collagenase II, a neutral non-clostridial protease, and any combination thereof.

In some kits, the defined culture medium comprises serum, for example, fetal bovine serum (FBS), at a concentration that ranges from about 1-5%, or that is about 1%, 2%, 3%, 4%, or 5%. In some kits, the frozen feeder cells are non-proliferating fibroblasts, for example, human fibroblasts.

Certain kits comprise supplemental SA, for example, to be added to the CTOS growth medium and/or the defined cell culture medium. In some embodiments, the CTOS growth medium and/or the defined cell culture medium comprise supplemental SA. In some embodiments, the supplemental SA is selected from bovine serum albumin (BSA) and human serum albumin (HSA). In certain embodiments, the concentration of the supplemental SA in the CTOS growth medium and/or the defined cell culture medium is from about 0.5 mg/ml to about 20 mg/ml, or about, less than about, or no more than about 0.5, 0.6, 0.7, 0.8, 0.9, 1.0, 1.2, 1.4, 1.6, 1.8, 2.0, 2.2, 2.5, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, 10.0, 10.2, 10.4, 10.6, 10.8, 11.0, 11.2, 11.4, 11.6, 11.8, 12.0, 12.2, 12.4, 12.6, 12.8, 13.0, 13.2, 13.4, 13.6, 13.8, 14.0, 14.2, 14.4, 14.6, 14.8, 15.0, 15.2, 15.4, 15.6, 15.8, 16.0, 16.2, 16.4, 16.6, 16.8, 17.0, 17.2, 17.4, 17.6, 17.8, 18.0, 18.2, 18.4, 18.6, 18.8, 19.0, 19.2, 19.4, 19.6, 19.8, or 20 mg/ml.

In some kits, the ROCK inhibitor is selected from one or more of Y27632, HA1100 hydrochloride, HA1077, and GSK429286. In some kits, the cellular proliferation marker is selected from one or more of ³H-thymidine, bromodeoxyuridine (BrdU), 5-ethynyl-2′-deoxyuridine (Edu), Ki-67, and proliferating cell nuclear antigen (PCNA). In some kits, the cellular apoptosis marker is selected from one or more of fluorochrome-labeled inhibitors of Caspases (FLICA), caspase activation, poly ADP ribose polymerase (PARP) cleavage, DRAQ5, DRAQ7, and terminal deoxynucleotidyl transferase (TdT) dUTP nick-end labeling (TUNEL) assay.

### BRIEF DESCRIPTION OF THE DRAWINGS

FIGS. 1A-1D show tumor cells that were isolated from patient colorectal tumor surgical samples, and cultured in suspension in a defined growth media to form cancer-tissue originated spheroids (CTOSs).

FIGS. 2A-2B show single cells that were isolated from normal colorectal tissue surgical samples.

FIGS. 3A-3B show cells isolated from normal colorectal tissue surgical samples, and seeded in a feeder cell/Rock inhibitor (Y27632) co-culture system. Cell proliferation was measured by Edu incorporation. Blue staining is DAPI, yellow staining is EpCam, and red staining is Edu.

FIGS. 4A-4F shows single tumor cells from six different patient-derived, cancer-tissue originated spheroids (CTOSs)) that were dissociated and seeded in a feeder cell/Rock inhibitor (Y27632) selective co-culture system. Cell proliferation was measured by Edu incorporation. Blue staining is DAPI, yellow staining is EpCam, and red staining is Edu.

FIGS. 5A-5D show non-enriched single cells that were isolated from patient colorectal tumor surgical samples, and seeded in feeder cell/Rock inhibitor (Y27632) selective co-culture system. Cell proliferation was measured by Edu incorporation.

FIG. 6 shows a post-enrichment co-culture system in which the primary epithelial tumor cells were used to test responsiveness to drugs and drug combinations. Blue staining is DAPI, yellow staining is EpCam, and red staining is Edu. Top to bottom rows are as follows: Fluorouracil (5-FU); oxaliplatin; oxaliplatin+5-FU (10 μM); SN-38; SN-38+5-FU (10 μM); SN-38+5-FU (10 μM)+oxaliplatin (2.5 μM) Columns from left to right show decreasing concentration of primary drug tested.

FIGS. 7A-7B show response profiles of epithelial tumor cells from eight individual patients to certain chemotherapeutics and combinations thereof, including tumor growth inhibition at AUC (7A) and quantitative drug sensitivity scores (7B).

FIGS. 8A-8F show cells isolated from normal colorectal tissue surgical samples from two different patients, seeded, and grew in a feeder cell/Rock inhibitor (Y27632) co-culture system with (8A-8B) complete medium, (8C-8D) complete medium w/o Wnt-3A-Noggin, (8E-8F) complete medium w/o R-Spondin and Noggin, and (8G-8H) complete medium w/o Wnt-3A, R-Spondin, and Noggin. Cell proliferation was measured by Edu incorporation. Blue staining is DAPI, yellow staining is EpCam, and red staining is Edu. Normal colorectal epithelial cells do not grow in the selective medium of FIGS. 8E-8F or the selective medium of FIGS. 8G-8H.

FIGS. 9A-9F shows tumor cells isolated from patient-derived, cancer-tissue originated spheroids (CTOSs) that were dissociated and seeded in a feeder cell/Rock inhibitor (Y27632) co-culture system (9A-9B) complete medium, (9C-9D) complete medium w/o Wnt-3A-Noggin, (9E-9F) complete medium w/o R-Spondin and Noggin, and (9G-9H) complete medium w/o Wnt-3A, R-Spondin, and Noggin. Cell proliferation was measured by Edu incorporation. Blue staining is DAPI, yellow staining is EpCam, and red staining is Edu. In contrast to normal cells, tumor colorectal epithelial cells grow in the selective medium of FIGS. 9E-9F and the selective medium of FIGS. 9G-9H.

FIG. 10 shows that supplemental bovine serum albumin (BSA) supports the growth of human epithelial tumor cells. DF12: DMEM/F12 based culture medium. DF12+BSA: DMEM/12 based culture medium supplemented with BSA. Cell proliferation was measured by EdU incorporation. Error bars represent standard deviation (SD).

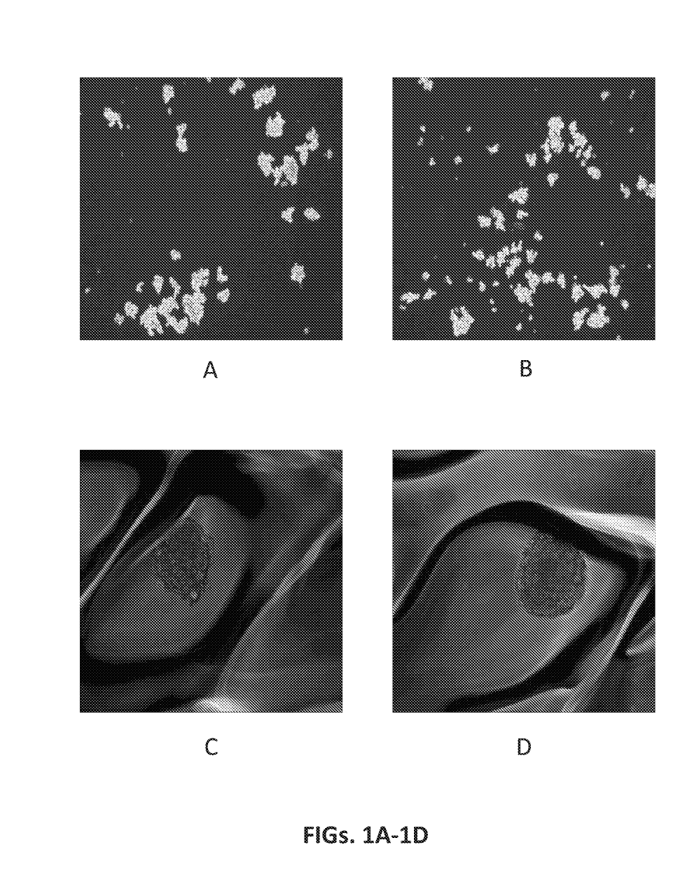

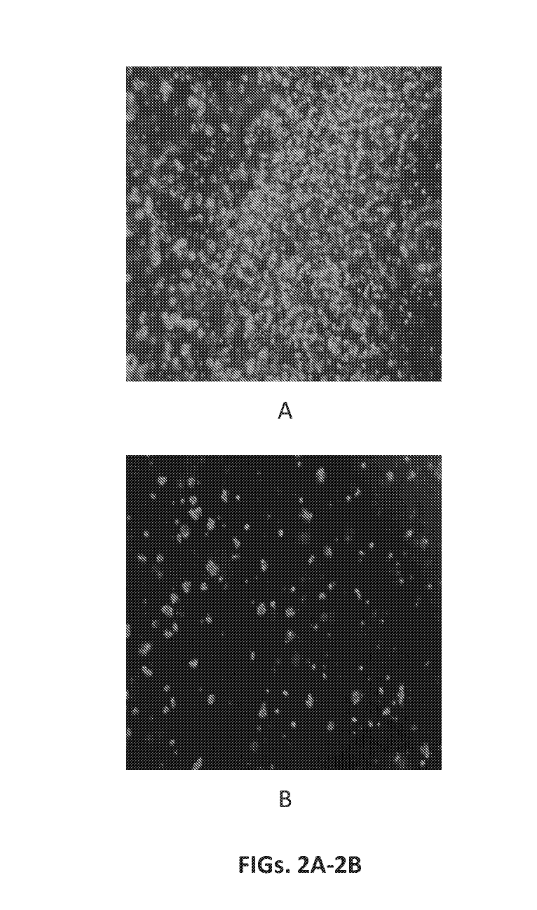

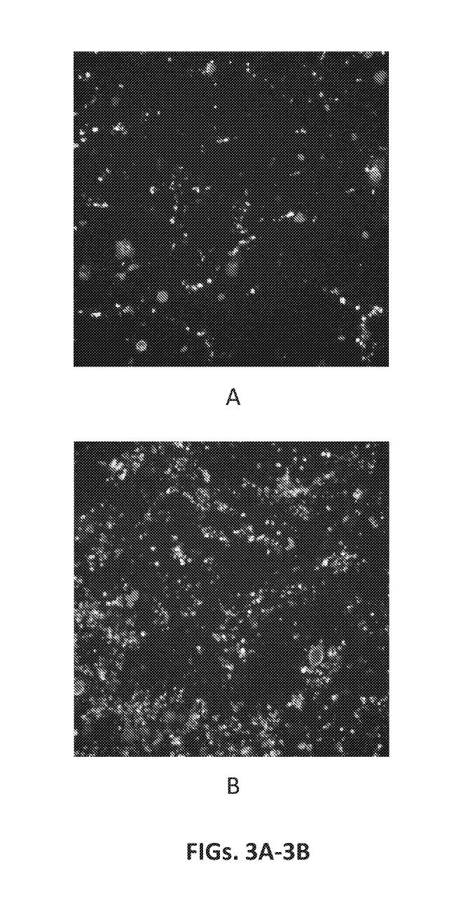

### DETAILED DESCRIPTION

Embodiments of the present disclosure relate to the discovery of methods and compositions for enriching patient-derived epithelial tumor cells by conditional reprogramming of cancer-tissue originated spheroids (CTOSs). The methods and compositions described herein not only reduce stromal cell overgrowth in feeder cell co-cultures but also maintain the clonal diversity of the original tumor sample. These methods and compositions allow the timely purification and proliferation of primary tumor cells derived directly from patients, and can be used, for example, to evaluate the potential responsiveness of patients tumor cells to one of more therapeutic agents. Such provides the advantage of identifying optimal treatment options for the patient in a relatively short time frame, for example, in less than one or two weeks from the time of initial culture.

Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, certain preferred methods and materials are described herein. All publications and references, including but not limited to patents and patent applications, cited in this specification are incorporated by reference in their entireties as if each individual publication or reference were specifically and individually indicated to be incorporated by reference. For the purposes of the present invention, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

By “isolated” is meant material that is substantially or essentially free from components that normally accompany it in its native state.

The term “modulating” includes “increasing” or “enhancing,” as well as “decreasing” or “reducing,” typically in a statistically significant or a physiologically significant amount as compared to a control. An “increased” or “enhanced” amount is typically a “statistically significant” amount, and may include an increase that is about or at least about 1.2, 1.4, 1.6, 1.8, 2, 3, 4, 5, 6, 7, 8, 9, 10, 15, 20, 30, 50, 60, 70, 80, 90, 100, 200, 300, 400, 500, 1000 times, or about or at least about 5%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, the amount produced by no composition or a control composition, sample, or test subject (including all integers and ranges in between). A “decreased” or “reduced” amount is typically a “statistically significant” amount, and may include a decrease that is about or at least about 1.2, 1.4, 1.6, 1.8, 2, 3, 4, 5, 6, 7, 8, 9, 10, 15, 20, 30, 50, 60, 70, 80, 90, 100, 200, 300, 400, 500, 1000 times, or about or at least about 5%, 10%, 11%, 12%, 13%, 14%, 15%, 16%, 17%, 18% , 19%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, the amount produced by no composition or a control composition, sample, or test subject (including all integers and ranges in between).

In certain embodiments, the “purity” of the epithelial tumor cells in a cell culture may be specifically defined. For instance, certain media or cultures may comprise epithelial tumor cells that are about or at least about 50%, 55%, 60%, 65%, 70%, 75%, 80%, 85%, 90%, 95%, 100% pure, including all integers and ranges in between), relative to other patient cell types, for example, non-cancerous cells such as stromal cells.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance, 95%, 96%, 97%, 98%, 99% or greater of some given quantity.

As noted above, certain embodiments relate to a cell culture medium and related co-cultures and methods for conditional reprogramming of cancer-tissue originated spheroids that have been dissociated into substantially single cell suspensions. The term “cancer-tissue originated spheroid” (or “CTOS”) refers to spheroid clusters of primary epithelial tumor cells in which cell-cell contact is retained between the highly purified and viable tumor cells (see, for example, Kondo et al., PNAS USA. 108:6235-6240, 2011; Endo et al., J Thorac Oncol. 8:131-9, 2013). Exemplary methods for preparing CTOSs are known in the art (supra) and described herein. Cell culture conditions for conditional reprogramming of epithelial cells are described, for example, in WO 2012065067 and Liu et al., Am J Pathol. 180:599-607, 2012).

Certain embodiments thus relate to a cell culture medium, comprising (a) human ex vivo-derived cancer-tissue originated spheroids (CTOSs) which are dissociated into a substantially single cell suspension, wherein the CTOSs comprise human epithelial tumor cells, (b) feeder cells; and (c) a defined cell culture medium that comprises at least one Rho-associated, coiled-coil containing protein kinase (ROCK) inhibitor; wherein the cell culture medium provides at least about 10% proliferation of the epithelial tumor cells within about 14 days following co-culture of the dissociated CTOSs with (b) and (c). In some embodiments, the cell culture medium does not comprise or is substantially free of a Bone Morphogenetic Protein (BMP) inhibitor such as Noggin and a Wnt/β-catenin signaling agonist such as R-spondin-1. In some embodiments, the cell culture medium comprises supplemental serum albumin (SA). The term “supplemental” refers to SA that differs from, is heterologous to, or is in addition to, SA that is present in any given animal serum, for example, fetal bovine serum (FBS) or fetal calf serum (FCS).

Also included are methods of culturing or expanding human epithelial tumor cells in an in vitro cell culture medium, comprising (A) dissociating human ex vivo-derived cancer-tissue originated spheroids (CTOSs) which comprise the human epithelial tumor cells into a substantially single cell suspension; and (B) co-culturing the dissociated CTOSs with feeder cells in a defined cell culture medium that comprises at least one Rho-associated, coiled-coil containing protein kinase (ROCK) inhibitor.

In some embodiments, the co-culture or growth medium does not comprise or is substantially free of further factors or supplements, for example, exogenous factors or supplements, such as nicotinamide, Wnt3A, Bone Morphogenetic Protein (BMP) inhibitors such as Noggin, Wnt/β-catenin signaling agonists such as R-spondin-1, and any combination of the foregoing.

In some embodiments, the CTOSs are cultured from a tumor-containing sample removed from a human patient with cancer. Non-limiting examples of tumor-containing samples include surgical samples, biopsy samples, pleural effusion samples, ascetic fluid samples, among others known in the art. In particular embodiments, the human patient has a cancer selected from colon cancer, lung cancer, gastric cancer, and breast cancer, and the human epithelial tumor cells are selected from colon cancer cells, lung cancer cells, gastric cancer cell, and breast cancer cells.

In certain embodiments, the epithelial tumor cells substantially retain the clonal diversity (for example, about or at least about 90%, 95%, 96%, 97%, 98%, 99%) of the tumor-containing sample removed from the human patient within about 14, 13, 12, 11, 10, 9, 8, 7, 6, 5, 4, 3, 2, or 1 days (including all combinations, integers, and ranges in between) days following co-culture of the dissociated CTOSs with (b) and (c). Clonal diversity of a population of tumor cells can be measured according to techniques known in the art (see, for example, Shibata, Nature Genetics. 38:402-403, 2006; Maley et al., Nature Genetics. 38:468-473, 2006; and Merlo et al., Cancer Prey Res. 3:1388-1397, 2010).

In some embodiments, the cell culture medium and methods provide about or at least about 10, 20, 30, 40, 50, 60, 70, 80, 90, or 100% proliferation of the epithelial tumor cells within about 14, 13, 12, 11, 10, 9, 8, 7, 6, 5, 4, 3, 2, or 1 days (including all combinations, integers, and ranges in between) following co-culture of the dissociated CTOSs with (b) and (c) or following steps (A) and (B). For example, particular cell culture media and methods provide about or at least about 10-15, 10-20, 10-25, 10-30, 10-35, 10-40, 10-45, 10-50, 10-55, 10-60, 10-65, 10-70, 10-75, 10-80, 10-85, 10-90, 10-95, or 10-100% proliferation of the epithelial tumor cells, or about or at least about 15-20, 15-25, 15-30, 15-35, 15-40, 15-45, 15-50, 15-55, 15-60, 15-65, 15-70, 15-75, 15-80, 15-85, 15-90, 15-95, or 15-100% proliferation of the epithelial tumor cells, or about or at least about 20-25, 20-30, 20-35, 20-40, 20-45, 20-50, 20-55, 20-60, 20-65, 20-70, 20-75, 20-80, 20-85, 20-90, 20-95, or 20-100% proliferation of the epithelial tumor cells, or about or at least about 25-30, 25-35, 25-40, 25-45, 25-50, 25-55, 25-60, 25-65, 25-70, 25-75, 25-80, 25-85, 25-90, 25-95, or 25-100% proliferation of the epithelial tumor cells, or about or at least about 30-35, 30-40, 30-45, 30-50, 30-55, 30-60, 30-65, 30-70, 30-75, 30-80, 30-85, 30-90, 30-95, or 30-100% proliferation of the epithelial tumor cells, or about or at least about 35-40, 35-45, 35-50, 35-55, 35-60, 35-65, 35-70, 35-75, 35-80, 35-85, 35-90, 35-95, or 35-100% proliferation of the epithelial tumor cells, or about or at least about 40-45, 40-50, 40-55, 40-60, 40-65, 40-70, 40-75, 40-80, 40-85, 40-90, 40-95, or 40-100% proliferation of the epithelial tumor cells, or about or at least about 45-50, 45-55, 45-60, 45-65, 45-70, 45-75, 45-80, 45-85, 45-90, 45-95, or 45-100% proliferation of the epithelial tumor cells, or about or at least about 50-55, 50-60, 50-65, 50-70, 50-75, 50-80, 50-85, 50-90, 50-95, or 50-100% proliferation of the epithelial tumor cells, or about or at least about 55-60, 55-65, 55-70, 55-75, 55-80, 55-85, 55-90, 55-95, or 55-100% proliferation of the epithelial tumor cells, or about or at least about 60-65, 60-70, 60-75, 60-80, 60-85, 60-90, 60-95, or 60-100% proliferation of the epithelial tumor cells, or about or at least about 65-70, 65-75, 65-80, 65-85, 65-90, 65-95, or 65-100% proliferation of the epithelial tumor cells, or about or at least about 70-80, 70-85, 70-90, 70-95, or 70-100% proliferation of the epithelial tumor cells, or about or at least about 75-80, 75-85, 75-90, 75-95, or 75-100% proliferation of the epithelial tumor cells, or about or at least about 80-85, 80-90, 80-95, or 80-100% proliferation of the epithelial tumor cells, or about or at least about 85-90, 85-95, or 85-100% proliferation of the epithelial tumor cells, or about or at least about 90-95, or 90-100% proliferation of the epithelial tumor cells, or about or at least about 95-100% proliferation of the epithelial tumor cells, for example, within about 14, 13, 12, 11, 10, 9, 8, 7, 6, or 5 days, or within about 5-14, 6-14, 7-14, 8-14, 9-14, 10-14, 11-14, 12-14, or 13-14 days, or within about 5-13, 6-13, 7-13, 8-13, 9-13, 10-13, 11-13, or 12-13 days, or within about 5-12, 6-12, 7-12, 8-12, 9-12, 10-12, or 11-12 days, or within about 5-11, 6-11, 7-11, 8-11, 9-11, or 10-11 days, or within about 5-10, 6-10, 7-10, 8-10, or 9-10 days, or within about 5-9, 6-9, 7-9, or 8-9 days, or within about 5-8, 6-8, or 7-8 days, or within about 5-7 or 6-7 days, or within about 5-6 days following co-culture of the dissociated CTOSs with (b) and (c) or following steps (A) and (B), including all combinations thereof. Specific embodiments provide at least about 20-30% proliferation of the epithelial tumor cells within about 7 days, or at least about 60% proliferation of the epithelial tumor cells within about 7 days following co-culture of the dissociated CTOSs with (b) and (c) or following steps (A) and (B).

In some embodiments, the cell culture medium and methods provide a ratio of about or at least about 5:1, 10:1, 20:1, 50:1, 100:1, 200:1, 500:1, 1000:1 between the human epithelial tumor cells and human stromal cells within about 4, 13, 12, 11, 10, 9, 8, 7, 6, 5, 4, 3, 2, or 1 days (including all combinations, integers, and ranges in between) days following co-culture of the dissociated CTOSs with (b) and (c) or following steps (A) and (B). In some embodiments, the human epithelial tumor cells are characterized by cell morphology and/or cell surface expression of epithelial cell adhesion molecule (EpCAM) and/or CD133. In some embodiments, stromal cells are characterized by cell morphology and/or cell surface expression of vimentin and/or alpha-smooth muscle actin (SMA).

In some embodiments, the cell culture medium comprises a “defined medium.” In certain embodiments, a “defined medium” is a growth medium suitable for the in vitro or ex vivo cell culture of human or animal cells in which all of the chemical components are known. In some embodiments, the defined medium comprises serum, for example, fetal bovine serum (FBS) or fetal calf serum (FCS). In some embodiments, the concentration of the serum ranges from about 1-5%, or is about 1%, 2%, 3%, 4%, or 5%. In some embodiments, a defined medium is “serum-free” or “substantially serum-free,” that is, the medium lacks or substantially lacks added serum, for example, FBS or fetal calf serum FCS.

In some embodiments, the defined medium comprises a basal media such as DMEM, F12, RPMI 1640, StemPro® hESC SFM, or a combination thereof, for example, DMEM/F12, which is supplemented with additional components, for example, growth factors, antioxidants, and/or energy sources. Thus, in particular embodiments, the defined medium comprises Dulbecco's Modified Eagles Medium (DMEM), Ham's Nutrient Mixture (F12), RPMI 1640, DMEM/F12, or StemPro® hESC SFM. In some embodiments, the medium comprises DMEM/F12, for example, at a ratio of about 10:1, 9:1, 8:1, 7:1, 6:1, 5:1, 4:1. 3:1, 2:1, 1:1, 1:2, 1:3, 1:4, 1:5, 1:6, 1:7, 1:8, 1:9, 1:10. In some embodiments, as noted above, the cell culture medium comprises, or the methods utilize, a ROCK inhibitor, for example, a ROCK-1 and/or a ROCK-2 inhibitor. Non-limited examples of ROCK inhibitors include Y27632, HA1100 hydrochloride, HA1077, and GSK429286. In particular embodiments, the concentration of the ROCK inhibitor (for example, Y27632) ranges from about 0.1-1000, 0.5-1000, 1-1000, 5-1000, 10-1000, 50-1000, 100-1000, 500-1000 μM, or from about 0.1-500, 0.5-500, 1-500, 5-500, 10-500, 50-500, 100-500 μM, or from about 0.1-100, 0.5-100, 1-100, 5-100, 10-100, 50-100 μM, or from about 0.1-50, 0.5-50, 1-50, 5-50, 10-50 μM, or from about 0.1-40, 0.5-40, 1-40, 5-40, 10-40 μM, or from about 0.1-30, 0.5-30, 1-30, 5-30, 10-30 μM, or from about 0.1-20, 0.5-20, 1-20, 5-20, 10-20 μM, or from about 0.1-10, 0.5-10, 1-10, 5-10 μM, or from about 0.1-5, 0.5-5, 1-5 μM, or from about 0.1-1 or 0.5-1 μM.

In some embodiments, as noted above, the cell culture medium comprises, or the methods utilize, supplemental SA, such as BSA and/or HAS. In certain embodiments, the concentration of the supplemental SA is from about 0.5 mg/ml to about 20 mg/ml, or about, less than about, or no more than about 0.5, 0.6, 0.7, 0.8, 0.9, 1.0, 1.2, 1.4, 1.6, 1.8, 2.0, 2.2, 2.5, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, 10.0, 10.2, 10.4, 10.6, 10.8, 11.0, 11.2, 11.4, 11.6, 11.8, 12.0, 12.2, 12.4, 12.6, 12.8, 13.0, 13.2, 13.4, 13.6, 13.8, 14.0, 14.2, 14.4, 14.6, 14.8, 15.0, 15.2, 15.4, 15.6, 15.8, 16.0, 16.2, 16.4, 16.6, 16.8, 17.0, 17.2, 17.4, 17.6, 17.8, 18.0, 18.2, 18.4, 18.6, 18.8, 19.0, 19.2, 19.4, 19.6, 19.8, or 20 mg/ml.

In particular embodiments, the cell culture medium comprises, or the methods utilize, feeder cells. As used herein, “co-culturing with dissociated CTOSs” means that the feeder cells share the same medium and the same vessel or culture plate as the CTOSs. In some embodiments, the feeder cells are non-proliferating feeder cells. For example, in some instances the feeder cells are treated to inhibit proliferation but maintain metabolic activity. In specific instances, the feeder cells are irradiated with gamma irradiation and/or treated with mitomycin C, which arrests cell division but maintains metabolic activity of the cells. In some embodiments, the feeder cells are not treated to inhibit proliferation. For example, in some instances feeder cells are placed on a porous filter that prevents physical contact with the CTOSs, and are cultured with the CTOSs without the need to treat the feeder cells to inhibit their proliferation.

Feeder cells can be obtained from any mammal and the animal source of the feeder cells need not be the same animal source as the CTOSs being cultured. For example, exemplary sources of feeder cells included, but are not limited to, mouse, rat, canine, feline, bovine, equine, porcine, non-human primate, and human feeder cells. Specific types of feeder cells include splenocytes, macrophages, thymocytes, and fibroblasts. In some embodiment, the splenocytes, macrophages, thymocytes, and/or fibroblasts are non-proliferating. One specific example includes J2 cells, which are a subclone of mouse fibroblasts derived from the established Swiss 3T3 cell line. In some embodiment, the J2 cells are gamma irradiated. In particular embodiment, the J2 cells are treated with mitomycin C. In specific embodiments, the feeder cells are non-proliferating fibroblasts, for example, human fibroblasts.

In certain embodiments, the cell culture medium or the methods exclude or otherwise do not comprise an extracellular matrix (ECM) component. Particular examples of ECMs include collagen, matrigel, laminin, and fibronectin.

In some embodiments, the co-cultures of human ex vivo-derived CTOSs which are dissociated into a substantially single cell suspension and the feeder cells are cultured in low cell-binding plates, or tissue culture plates having a low cell-binding surface. Non-limited examples include 6-well, 12-well, 24-well, 48-well plates and microtiter plates such as 96-well, 384-well, and 1536-well plates, which have a low cell-binding surface. Low cell binding plates are commercially available (see, for example, Nunc™ Low Cell Binding Plates or Microplates). The use of low cell-binding plates can, for example, reduce cell attachment and unwanted differentiation of stem cells, and preferentially deplete normal epithelial cells and stromal cells relative to epithelial tumor cells. Thus, certain embodiments relate to a tissue culture plate having a low cell binding surface, comprising a cell culture medium or co-culture described herein. Certain methods employ a tissue culture plate having a low cell binding surface, for example, for the co-culturing step or steps.

In certain embodiments, as noted above, the human ex vivo-derived CTOSs are cultured from a tumor sample removed from a human patient with cancer, which comprises mincing and incubating the tumor-containing sample in a tissue digestion medium, and culturing the tumor-containing sample in a defined, serum-free (or substantially serum-free), and feeder-free CTOS growth medium that comprises nicotinamide, Wnt3A, a Bone Morphogenetic Protein (BMP) inhibitor, a Wnt/β-catenin signaling agonist, and a ROCK inhibitor, for a time sufficient to form the CTOSs. In specific embodiments, the CTOS growth medium is StemPro® hESC SFM (defined, serum- and feeder-free medium (SFM)) supplemented with: nicotinamide, Wnt3A, Noggin, R-spondin-1, and Y27632. In specific embodiments, the CTOS growth medium is StemPro® hESC SFM (defined, serum- and feeder-free medium (SFM)) supplemented with: nicotinamide, Wnt3A, and Y27632, and which does not comprise or is substantially free of Noggin, R-spondin-1. In specific embodiments, the CTOS growth medium is StemPro® hESC SFM (defined, serum- and feeder-free medium (SFM)) supplemented with: nicotinamide and Y27632, and which does not comprise or is substantially free of Noggin, R-spondin-1, and Wnt3A.

In some embodiments, the tissue digestion or tissue dissociation medium comprises one or more proteases. Examples of proteases include collagenase I, collagenase II, a neutral non-clostridial protease, and any combination thereof. Non-limiting examples of commercially-available tissue digestion or dissociation media include Liberase™, Liberase™ TL, Liberase™ TM, and Liberase™ DH, and others.

In some embodiments, the concentration of nicotinamide in the CTOS growth medium ranges from about 0.1-1000, 0.5-1000, 1-1000, 5-1000, 10-1000, 50-1000, 100-1000, 500-1000 mM, or from about 0.1-500, 0.5-500, 1-500, 5-500, 10-500, 50-500, 100-500 mM, or from about 0.1-100, 0.5-100, 1-100, 5-100, 10-100, 50-100 mM, or from about 0.1-50, 0.5-50, 1-50, 5-50, 10-50 mM, or from about 0.1-40, 0.5-40, 1-40, 5-40, 10-40 mM, or from about 0.1-30, 0.5-30, 1-30, 5-30, 10-30 mM, or from about 0.1-20, 0.5-20, 1-20, 5-20, 10-20 mM, or from about 0.1-10, 0.5-10, 1-10, 5-10 mM, or from about 0.1-5, 0.5-5, 1-5 mM, or from about 0.1-1 or 0.5-1 mM.

In certain embodiments, the concentration of Wnt3A in the CTOS growth medium ranges from about 0.1-10,000 or 1-1000 ng/ml, or from about 0.1-1000, 1-1000, 10-1000, 20-1000, 30-1000, 40-1000, 50-1000, 60-1000, 70-1000, 80-1000, 90-1000, 100-1000, 200-1000, 300-1000, 400-1000, 500-1000, 600-1000, 700-1000, 800-1000, 900-1000 ng/ml, or from about 0.1-500, 1-500, 10-500, 20-500, 30-500, 40-500, 50-500, 60-500, 70-500, 80-500, 90-500, 100-500, 200-500, 300-500, 400-500 ng/ml, or from about 0.1-400, 1-400, 10-400, 20-400, 30-400, 50-400, 40-400, 60-400, 70-400, 80-400, 90-400, 100-400, 200-400, 300-400 ng/ml, or from about 0.1-300, 1-300, 10-300, 20-300, 30-300, 40-300, 50-300, 60-300, 70-300, 80-300, 90-300, 100-300, 200-300 ng/ml, or from about 0.1-200, 1-200, 10-200, 20-200, 30-200, 40-200, 50-200, 60-200, 70-200, 80-200, 90-200, 100-200 ng/ml, or from about 0.1-150, 1-150, 10-150, 20-150, 30-150, 40-150, 50-150, 60-150, 70-150, 80-150, 90-150, 100-150 ng/ml, or from about 0.1-120, 1-120, 10-120, 20-120, 30-120, 40-120, 50-120, 60-120, 70-120, 80-120, 90-120, 100-120 ng/ml, or from about 0.1-100, 1-100, 10-100, 20-100, 30-100, 40-100, 50-100, 60-100, 70-100, 80-100, 90-100 ng/ml.

In some embodiments, the BMP inhibitor in the CTOS growth medium is noggin. In particular embodiments, the concentration of the BMP inhibitor, for example, noggin, ranges from about 0.1-10,000 or 1-1000 ng/ml, or from about 0.1-1000, 1-1000, 10-1000, 20-1000, 30-1000, 40-1000, 50-1000, 60-1000, 70-1000, 80-1000, 90-1000, 100-1000, 200-1000, 300-1000, 400-1000, 500-1000, 600-1000, 700-1000, 800-1000, 900-1000 ng/ml, or from about 0.1-500, 1-500, 10-500, 20-500, 30-500, 40-500, 50-500, 60-500, 70-500, 80-500, 90-500, 100-500, 200-500, 300-500, 400-500 ng/ml, or from about 0.1-400, 1-400, 10-400, 20-400, 30-400, 50-400, 40-400, 60-400, 70-400, 80-400, 90-400, 100-400, 200-400, 300-400 ng/ml, or from about 0.1-300, 1-300, 10-300, 20-300, 30-300, 40-300, 50-300, 60-300, 70-300, 80-300, 90-300, 100-300, 200-300 ng/ml, or from about 0.1-200, 1-200, 10-200, 20-200, 30-200, 40-200, 50-200, 60-200, 70-200, 80-200, 90-200, 100-200 ng/ml, or from about 0.1-150, 1-150, 10-150, 20-150, 30-150, 40-150, 50-150, 60-150, 70-150, 80-150, 90-150, 100-150 ng/ml, or from about 0.1-120, 1-120, 10-120, 20-120, 30-120, 40-120, 50-120, 60-120, 70-120, 80-120, 90-120, 100-120 ng/ml, or from about 0.1-100, 1-100, 10-100, 20-100, 30-100, 40-100, 50-100, 60-100, 70-100, 80-100, 90-100 ng/ml.

In particular embodiments, the Wnt/β-catenin signaling agonist in the CTOS growth medium is R-spondin-1. In some embodiments, the concentration of the Wnt/β-catenin signaling agonist, for example, R-spondin-1, ranges from about 0.1-10,000 or 1-1000 or 100-1000 ng/ml, or from about 0.1-10,000, 1-10,000, 10-10,000, 20-10,000, 30-10,000, 40-10,000, 50-10,000, 60-10,000, 70-10,000, 80-10,000, 90-10,000, 100-10,000, 200-10,000, 300-10,000, 400-10,000, 500-10,000, 1000-10,000, 5000-10,000 ng/ml, or from about 0.1-5000, 1-5000, 10-5000, 20-5000, 30-10,000, 40-5000, 50-5000, 60-5000, 70-5000, 80-5000, 90-5000, 100-5000, 200-5000, 300-5000, 400-5000, 500-5000, 1000-5000 ng/ml, or from about 0.1-1000, 1-1000, 10-1000, 20-1000, 30-1000, 40-1000, 50-1000, 60-1000, 70-1000, 80-1000, 90-1000, 100-1000, 200-1000, 300-1000, 400-1000, 500-1000, 600-1000, 700-1000, 800-1000, 900-1000 ng/ml, or from about 0.1-800, 1-800, 10-800, 20-800, 30-800, 40-800, 50-800, 60-800, 70-800, 80-800, 90-800, 100-800, 200-800, 300-800, 400-800, 500-800, 600-800, 700-800 ng/ml, or from about 0.1-700, 1-700, 10-700, 20-700, 30-700, 40-700, 50-700, 60-700, 70-700, 80-700, 90-700, 100-700, 200-700, 300-700, 400-700, 500-700, 600-700 ng/ml, or from about 0.1-600, 1-600, 10-600, 20-600, 30-600, 40-600, 50-600, 60-600, 70-600, 80-600, 90-600, 100-600, 200-600, 300-600, 400-600, 500-600 ng/ml, or from about 0.1-500, 1-500, 10-500, 20-500, 30-500, 40-500, 50-500, 60-500, 70-500, 80-500, 90-500, 100-500, 200-500, 300-500, 400-500 ng/ml.

In some embodiments, the ROCK inhibitor in the CTOS growth medium is Y27632. In some embodiments, the concentration of the ROCK inhibitor, for example, Y27632, ranges from about 0.1-1000, 0.5-1000, 1-1000, 5-1000, 10-1000, 50-1000, 100-1000, 500-1000 μM, or from about 0.1-500, 0.5-500, 1-500, 5-500, 10-500, 50-500, 100-500 μM, or from about 0.1-100, 0.5-100, 1-100, 5-100, 10-100, 50-100 μM, or from about 0.1-50, 0.5-50, 1-50, 5-50, 10-50 μM, or from about 0.1-40, 0.5-40, 1-40, 5-40, 10-40 μM, or from about 0.1-30, 0.5-30, 1-30, 5-30, 10-30 μM, or from about 0.1-20, 0.5-20, 1-20, 5-20, 10-20 μM, or from about 0.1-10, 0.5-10, 1-10, 5-10 μM, or from about 0.1-5, 0.5-5, 1-5 μM, or from about 0.1-1 or 0.5-1 μM.

In some embodiments, the digested or dissociated tumor-containing sample is cultured in the CTOS growth medium for about or less than about 6, 7, 8, 9, 10, 11, 12, 13, 14, 15, 16, 17, 18, 19, 20, 21, 22, 23, 24, 36, 48, 60, or 72 hours, or about or less than about 1, 2, 3, 4, 5, 6, or 7 days to form CTOSs, before being substantially dissociated into a single cell suspension and co-cultured with feeder cells and a ROCK inhibitor, as described herein.

As noted above, the cell culture media and methods described herein can be used, inter alia, to test or evaluate the potential responsiveness of patient tumors to one or more therapeutic agents, and provide the advantage of identifying optimal treatment options for the patient in a relatively short time frame, for example, in less than one or two weeks. Thus, certain embodiments include methods of testing responsiveness of a human patient to a therapeutic agent (e.g., a drug or candidate drug), comprising (a) administering the therapeutic agent to a cell culture medium described herein, or to a cell culture medium prepared by a method described herein; and (b) measuring epithelial tumor cell proliferation and/or epithelial tumor cell apoptosis.

In some embodiments, a decrease (e.g., statistically significant decrease) in epithelial tumor cell proliferation is indicative of responsiveness of the human patient (i.e., the tumor in the human patient) to the therapeutic agent, and in some embodiments, a lack of decrease (e.g., a lack of a statistically significant decrease) in epithelial tumor cell proliferation is indicative of resistance and likely non-responsiveness of the human patient (i.e., the tumor in the human patient) to the therapeutic agent. In some embodiments, an induction (e.g., statistically significant increase) in epithelial tumor cell apoptosis is indicative of responsiveness of the human patient (i.e., the tumor in the human patient) to the therapeutic agent. In particular embodiments, a lack of increase (e.g., a lack of a statistically significant increase) in epithelial tumor cell proliferation is indicative of resistance and likely non-responsiveness of the human patient (i.e., the tumor in the human patient) to the therapeutic agent. In certain embodiments, a decrease in epithelial tumor cell proliferation and an induction in tumor cell apoptosis is indicative of responsiveness of the human patient to the therapeutic agent, and a lack of decrease in epithelial tumor cell proliferation is indicative of resistance or likely non-responsiveness of the human patient to the therapeutic agent.

In some embodiments, the methods of testing responsiveness include administering the therapeutic agent on the same day (e.g., at the same time or substantially the same time) as dissociating and co-culturing CTOSs in the cell culture medium. Certain embodiment include administering the therapeutic agent within about or at about 1, 2, 3, 4, 5, 6, 7, 8, 9, 10, 11, 12, 13, 14, 15, 16, 17, 18, 19, 20, 21, 22, 23, 24, 36, 48, 60, or 72 hours of dissociating and co-culturing the CTOSs in the cell culture medium. Also included are methods of administering the therapeutic agent about or within about 1, 2, 3, 4, 5, 6, or 7 days after dissociating and co-culturing the CTOSs in the cell culture medium, including methods of administering the therapeutic agent about or within about 1-7, 1-6, 1-5, 1-4, 1-3, 1-2, or 2-7, 2-6, 2-5, 2-4, 2-3, or 3-7, 3-6, 3-5, 3-4, or 4-7, 4-6, 4-5, or 5-7, 5-6, or 6-7 days after dissociating and co- culturing the CTOSs in the cell culture medium.

Certain embodiments include the step of measuring tumor cell proliferation and/or tumor cell apoptosis in the population of tumor cells within about 14 days of administering the therapeutic agent, for example, within about 14, 13, 12, 11, 10, 9, 8, 7, 6, or 5 days of administering the therapeutic agent. Specific embodiments include the step of measuring tumor cell proliferation and/or tumor cell apoptosis in the population of tumor cells within about 5-14, 6-14, 7-14, 8-14, 9-14, 10-14, 11-14, 12-14, or 13-14 days, or within about 5-13, 6-13, 7-13, 8-13, 9-13, 10-13, 11-13, or 12-13 days, or within about 5-12, 6-12, 7-12, 8-12, 9-12, 10-12, or 11-12 days, or within about 5-11, 6-11, 7-11, 8-11, 9-11, or 10-11 days, or within about 5-10, 6-10, 7-10, 8-10, or 9-10 days, or within about 5-9, 6-9, 7-9, or 8-9 days, or within about 5-8, 6-8, or 7-8 days, or within about 5-7 or 6-7 days, or within about 5-6 days of administering the therapeutic agent.

In certain embodiments, the therapeutic agent or drug for testing is a small molecule. Exemplary small molecules include cytotoxic, chemotherapeutic, and anti-angiogenic agents, for instance, those that have been considered useful in the treatment of various cancers. Particular classes of small molecules include, without limitation, alkylating agents, anti-metabolites, anthracyclines, anti-tumor antibiotics, platinums, type I topoisomerase inhibitors, type II topoisomerase inhibitors, vinca alkaloids, and taxanes.

Specific examples of small molecules include chlorambucil, cyclophosphamide, cilengitide, lomustine (CCNU), melphalan, procarbazine, thiotepa, carmustine (BCNU), enzastaurin, busulfan, daunorubicin, doxorubicin, gefitinib, erlotinib idarubicin, temozolomide, epirubicin, mitoxantrone, bleomycin, cisplatin, carboplatin, oxaliplatin, camptothecins, irinotecan, topotecan, amsacrine, etoposide, etoposide phosphate, teniposide, temsirolimus, everolimus, vincristine, vinblastine, vinorelbine, vindesine, CT52923, and paclitaxel, and pharmaceutically acceptable salts, acids or derivatives of any of the above. Additional examples of small molecules include imatinib, crizotinib, dasatinib, sorafenib, pazopanib, sunitinib, vatalanib, geftinib, erlotinib, AEE-788, dichoroacetate, tamoxifen, fasudil, SB-681323, and semaxanib (SU5416) (see Chico et al., Nat Rev Drug Discov. 8:829-909, 2009).

Further examples of small molecules include alkylating agents such as thiotepa, cyclophosphamide (CYTOXANTM); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and docetaxel (TAXOTERE®, Rhne-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above.

Also included are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In some embodiments, the therapeutic agent is an antibody or an antigen-binding fragment thereof. In certain embodiments, the antibody or antigen-binding fragment thereof or other polypeptide specifically binds to a cancer-associated antigen, or cancer antigen, for example, a cancer antigen expressed by the tumor cell clusters being tested. Exemplary cancer antigens include cell surface proteins such as cell surface receptors. Also included as cancer-associated antigens are ligands that bind to such cell surface proteins or receptors. In specific embodiments, the antibody or antigen-binding fragment specifically binds to a intracellular cancer antigen. In some embodiments, the cancer that associates with the cancer antigen is one or more of breast cancer, metastatic brain cancer, prostate cancer, gastrointestinal cancer, lung cancer, ovarian cancer, testicular cancer, head and neck cancer, stomach cancer, bladder cancer, pancreatic cancer, liver cancer, kidney cancer, squamous cell carcinoma, CNS or brain cancer, melanoma, non-melanoma cancer, thyroid cancer, endometrial cancer, epithelial tumor, bone cancer, or a hematopoietic cancer.

In particular embodiments, the antibody or antigen-binding fragment or other polypeptide specifically binds to at least one cancer-associated antigen, or cancer antigen, such as human Her2/neu, Her1/EGF receptor (EGFR), Her3, A33 antigen, B7H3, CD5, CD19, CD20, CD22, CD23 (IgE Receptor), C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), integrin αᵥβ₃, integrin α₅β₁, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and/or mesothelin.

In certain embodiments, the antibody is a therapeutic antibody, such as an anti-cancer therapeutic antibody, including antibodies such as 3F8, 8H9, abagovomab, adecatumumab, afutuzumab, alemtuzumab, alacizumab (pegol), amatuximab, apolizumab, bavituximab, bectumomab, belimumab, bevacizumab, bivatuzumab (mertansine), brentuximab vedotin, cantuzumab (mertansine), cantuzumab (ravtansine), capromab (pendetide), catumaxomab, cetuximab, citatuzumab (bogatox), cixutumumab, clivatuzumab (tetraxetan), conatumumab, dacetuzumab, dalotuzumab, detumomab, drozitumab, ecromeximab, edrecolomab, elotuzumab, enavatuzumab, ensituximab, epratuzumab, ertumaxomab, etaracizumab, farletuzumab, FBTA05, figitumumab, flanvotumab, galiximab, gemtuzumab, ganitumab, gemtuzumab (ozogamicin), girentuximab, glembatumumab (vedotin), ibritumomab tiuxetan, icrucumab, igovomab, indatuximab ravtansine, intetumumab, inotuzumab ozogamicin, ipilimumab (MDX-101), iratumumab, labetuzumab, lexatumumab, lintuzumab, lorvotuzumab (mertansine), lucatumumab, lumiliximab, mapatumumab, matuzumab, milatuzumab, mitumomab, mogamulizumab, moxetumomab (pasudotox), nacolomab (tafenatox), naptumomab (estafenatox), narnatumab, necitumumab, nimotuzumab, nivolumab, Neuradiab® (with or without radioactive iodine), NR-LU-10, ofatumumab, olaratumab, onartuzumab, oportuzumab (monatox), oregovomab, panitumumab, patritumab, pemtumomab, pertuzumab, pritumumab, racotumomab, radretumab, ramucirumab, rilotumumab, rituximab, robatumumab, samalizumab, sibrotuzumab, siltuximab, tabalumab, taplitumomab (paptox), tenatumomab, teprotumumab, TGN1412, ticilimumab, tremelimumab, tigatuzumab, TNX-650, tositumomab, TRBS07, trastuzumab, tucotuzumab (celmoleukin), ublituximab, urelumab, veltuzumab, volociximab, votumumab, and zalutumumab. Also included are fragments, variants, and derivatives of these antibodies.

Certain embodiments include testing the responsiveness to combinations of two or more therapeutic agents. Thus, certain methods include administering two or more therapeutic agents to the medium suspension, including combinations of any two or more of the foregoing, exemplary therapeutic agents.

Any variety of methods known in the art can be used to measure tumor cell proliferation and/or tumor cell apoptosis. For example, certain methods of measuring tumor cell proliferation include measuring one or more cellular proliferation markers. Exemplary cellular proliferation markers include ³H-thymidine, bromodeoxyuridine (BrdU), 5-ethynyl-2′-deoxyuridine (Edu), Ki-67, and proliferating cell nuclear antigen (PCNA). Thus, in certain embodiments, the step of measuring tumor cell proliferation comprises measuring a cellular proliferation marker, which is selected, for example, from one or more of ³H-thymidine, BrdU, Edu, Ki-67, and PCNA, including combinations thereof.

Likewise, any variety of methods known in the art can be used to measure tumor cell apoptosis. “Apoptosis” refers generally to a process of programmed cell death that occurs in multicellular organisms, including biochemical events that lead to characteristic cell changes (e.g., morphology) and cell death. Exemplary changes include blebbing, cell shrinkage, nuclear fragmentation, chromatin condensation, chromosomal DNA fragmentation, and global mRNA decay. Certain methods of measuring tumor cell apoptosis include measuring a cellular apoptosis marker. Exemplary cellular apoptosis markers include fluorochrome-labeled inhibitors of Caspases (FLICA), caspase activation, poly ADP ribose polymerase (PARP) cleavage, DRAQS, DRAQ7, and a terminal deoxynucleotidyl transferase (TdT) dUTP nick-end labeling (TUNEL) assay. Thus, in certain embodiments, the step of measuring tumor cell apoptosis comprises measuring a cellular apoptosis marker, which is selected, for example, from one or more of FLICA, PARP, DRAQ5, DRAQ7, and a TUNEL assay, including combinations thereof.

In certain aspects, these responsiveness tests or methods are performed at a diagnostic laboratory, and the results are then provided to the patient, or to a physician or other healthcare provider that plays a role in the patient's healthcare and cancer treatment. Particular embodiments thus include methods for providing the results of the tumor cell cluster responsiveness test to the patient, or to the physician or other healthcare provider. These results or data can be in the form of a hard-copy or paper-copy, or an electronic form, such as a computer-readable medium.

Also included are kits comprising one or more of the various components described herein. For example, such kits can be employed to prepare co-cultures for the conditional reprogramming, and/or prepare CTOSs from a tumor-containing sample removed from a human patient with cancer.

Certain embodiments thus relate to a sample preparation kit, comprising any combination of:
a defined, serum-free, and feeder-free cancer-tissue originated spheroid (CTOSs) growth medium;
a tissue digestion or dissociation medium;
a tissue culture plate having a low cell binding surface;
a defined cell culture medium;
frozen feeder cells;
a Rho-associated, coiled-coil containing protein kinase (ROCK) inhibitor; and
a cellular proliferation marker and/or a cellular apoptosis marker.

In some embodiments, the kit provides, or the CTOS growth medium comprises, nicotinamide, Wnt3A, a Bone Morphogenetic Protein (BMP) inhibitor, a Wnt/β-catenin signaling agonist, and/or a ROCK inhibitor. Certain kits comprise or provide supplemental SA, for example, to be added to the CTOS growth medium and/or the defined cell culture medium. In some embodiments, the CTOS growth medium and/or the defined cell culture medium comprise supplemental SA. In some embodiments, the supplemental SA is selected from bovine serum albumin (BSA) and human serum albumin (HSA). In certain embodiments, the concentration of the supplemental SA in the CTOS growth medium and/or the defined cell culture medium is from about 0.5 mg/ml to about 20 mg/ml, or about, less than about, or no more than about 0.5, 0.6, 0.7, 0.8, 0.9, 1.0, 1.2, 1.4, 1.6, 1.8, 2.0, 2.2, 2.5, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, 10.0, 10.2, 10.4, 10.6, 10.8, 11.0, 11.2, 11.4, 11.6, 11.8, 12.0, 12.2, 12.4, 12.6, 12.8, 13.0, 13.2, 13.4, 13.6, 13.8, 14.0, 14.2, 14.4, 14.6, 14.8, 15.0, 15.2, 15.4, 15.6, 15.8, 16.0, 16.2, 16.4, 16.6, 16.8, 17.0, 17.2, 17.4, 17.6, 17.8, 18.0, 18.2, 18.4, 18.6, 18.8, 19.0, 19.2, 19.4, 19.6, 19.8, or 20 mg/ml.

In some embodiments, the defined culture medium comprises serum, for example, fetal bovine serum (FBS), at a concentration that ranges from about 1-5%, or that is about 1%, 2%, 3%, 4%, or 5%. In some embodiments, the ROCK inhibitor is selected from one or more of Y27632, HA1100 hydrochloride, HA1077, and GSK429286. In certain embodiments, the cellular proliferation marker is selected from one or more of ³H-thymidine, bromodeoxyuridine (BrdU), 5-ethynyl-2′-deoxyuridine (Edu), Ki-67, and proliferating cell nuclear antigen (PCNA). In particular embodiments, the cellular apoptosis marker is selected from one or more of fluorochrome-labeled inhibitors of Caspases (FLICA), caspase activation, poly ADP ribose polymerase (PARP) cleavage, DRAQ5, DRAQ7, and terminal deoxynucleotidyl transferase (TdT) dUTP nick-end labeling (TUNEL) assay.

The kits can also include written instructions, for example, on how to prepare CTOSs from patient samples, co-culture CTOSs for enrichment of tumor epithelial cells, and/or test the responsiveness of the tumor epithelial cells to one or more therapeutic agents.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill certain changes and modifications may be made thereto without departing from the spirit or scope of the description or appended claims. The following examples are provided by way of illustration only and not by way of limitation. Those of skill in the art will readily recognize a variety of noncritical parameters that could be changed or modified to yield essentially similar results.

### EXAMPLES

### Example 1

### Isolation of Patient-Derived Cancer-Tissue Originated Spheroids (CTOSs) and Conditional Reprogramming as Single Cell Cultures

Normal and tumor surgical samples from colorectal cancer patients were obtained from Beijing Tumor Hospital after receiving patient consensus.

Briefly, to prepare CTOS cultures, the non-tumor tissues and the necrotic fractions were carefully removed from the tumor specimen. Both normal and tumor tissues were cut into small pieces less than 1 mm in diameter using scissors. Minced tumor fractions were transferred into a sterile 100-ml triangle glass flask loaded with a magnet stir bar. A 10-15 ml digestion media containing 0.25 U/ml Liberase DH (collagenase I and II at defined ratio, with Dispase®, a non-clostridial neutral protease) was added into the minced tumor tissues to start enzyme digestion. The enzyme mixture was incubated at 37° C. for 1-2 hours with moderate stirring. The partial digested tumor cell clusters were filtered through a 100-μm cell retainer. The filtrates were re-filtered through a 40-μm cell restrainer. The tumor cell clusters retained on the 40-μm cell restrainer were collected, wash twice with HBSS, then re-suspended in a defined growth media supplemented with cell growth factors and small molecule inhibitors. The tumor cell clusters were recovered in a defined growth medium overnight. The defined complete growth medium was StemPro® hESC SFM (defined, serum- and feeder-free medium (SFM)) supplemented with: Nicotinamide, Wnt3A, Noggin (Bone Morphogenetic Protein (BMP) inhibitor), R-spondin-1 (Wnt/β-catenin signaling agonist), and Y27632 (Rho-associated, coiled-coil containing protein kinase (ROCK-1) inhibitor).

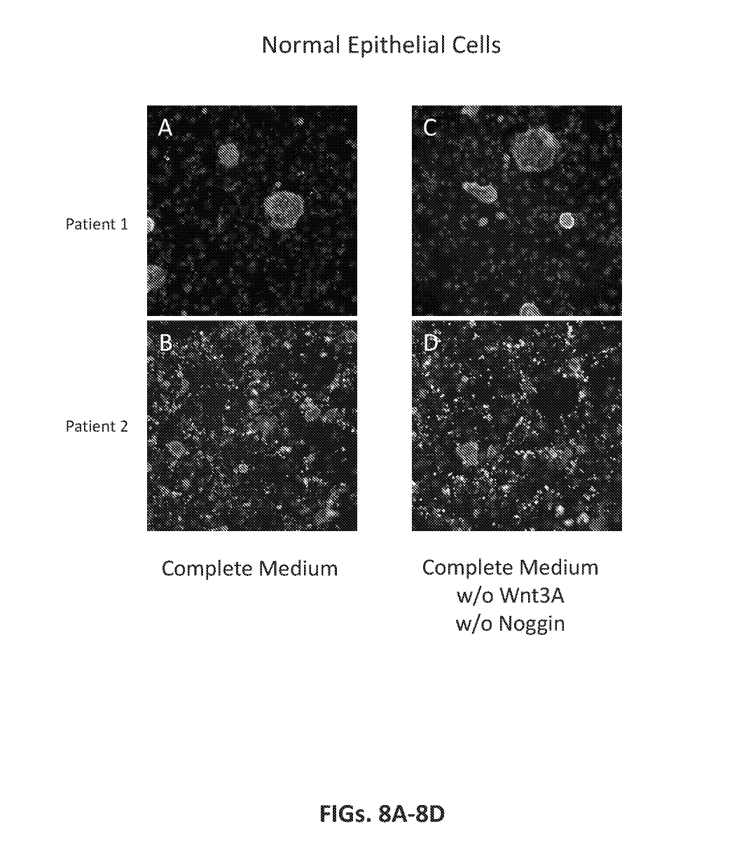

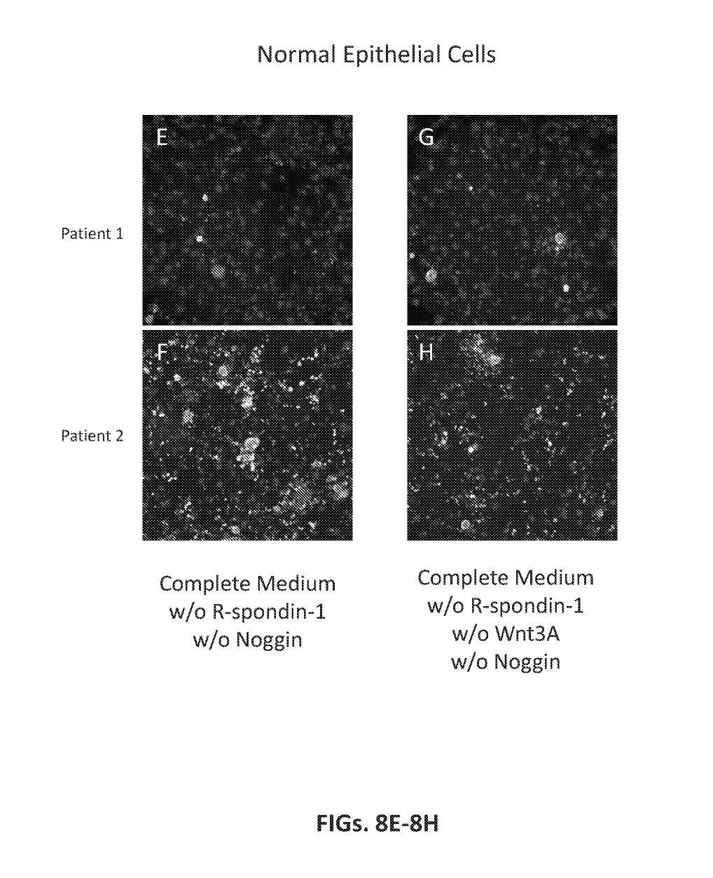

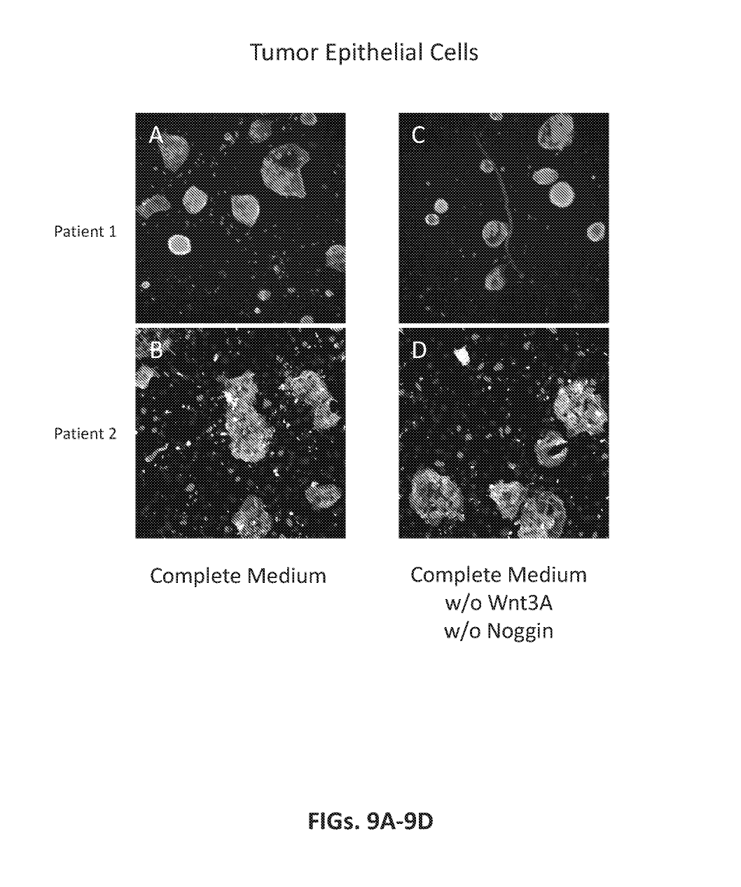

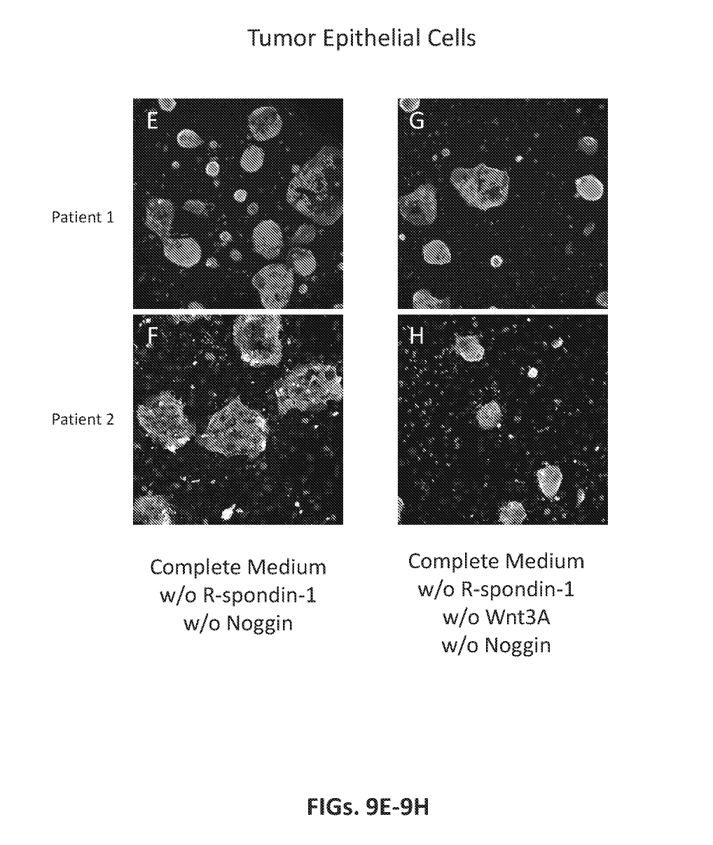

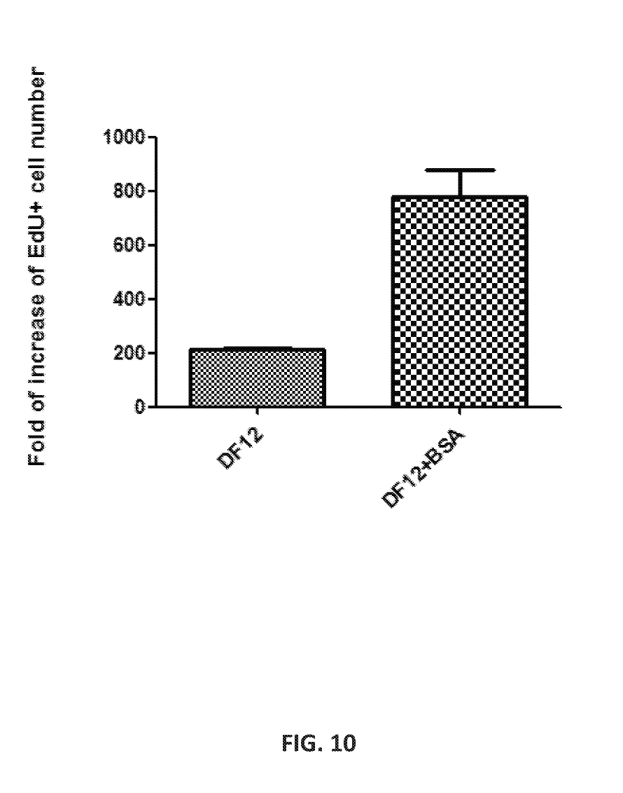

Selective growth media were also tested, which included the foregoing defined complete growth medium that was not supplemented with R-spondin-1 and Noggin, and in some instances was also not supplemented with Wnt3A (see FIGS. 8A-8H and FIGS. 9A-9H). Normal epithelial cells grew in the defined complete growth media (FIGS. 8A-8D) but did not grow in the selective growth media (FIGS. 8E-8H). In contrast, tumor epithelial cells grew in both the defined complete growth media (FIGS. 9A-9D) and the selective growth media (FIGS. 9E-9H).

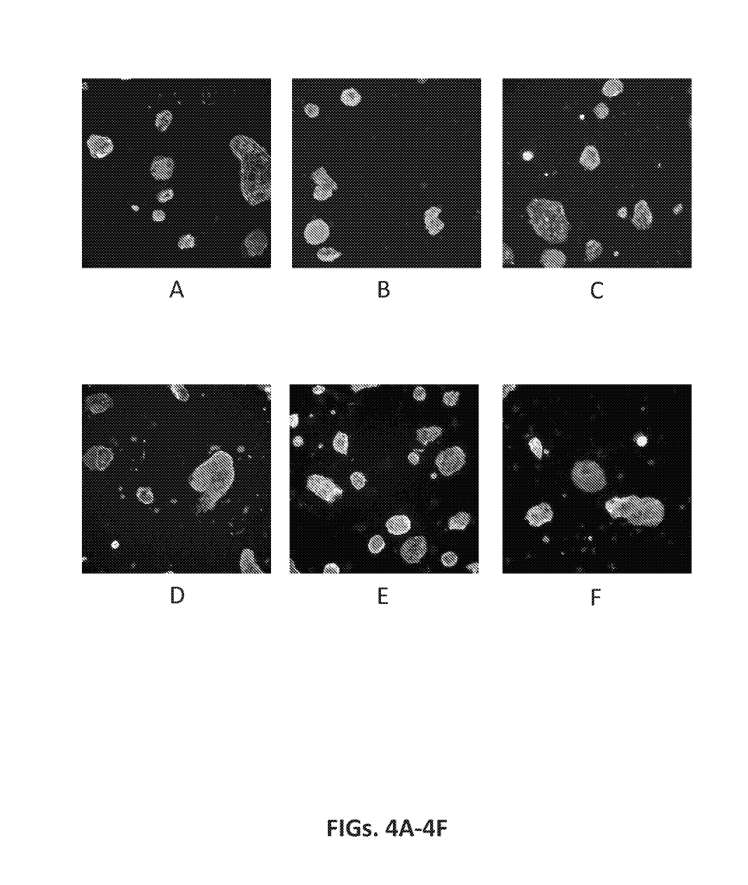

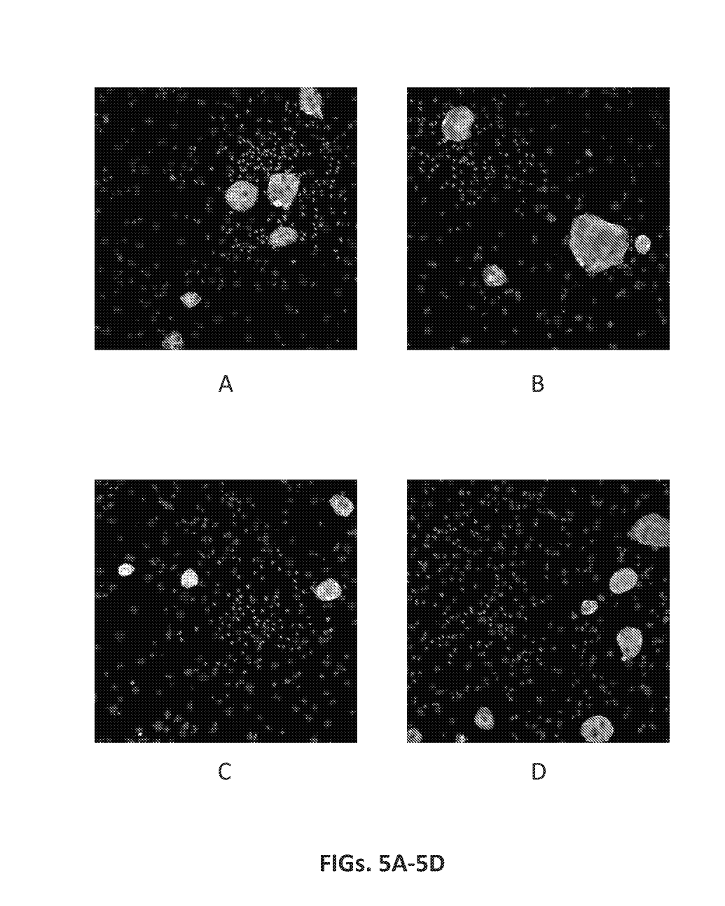

For conditional reprogramming, the recovered CTOSs were dissociated and seeded in low-cell binding plates with a feeder cell/Rock inhibitor (Y27632) co-culture system at about 3000 cells per well (see FIGS. 4A-4F).

After initial seeding, the dissociated tumor epithelial cells were used for drug testing in a defined selective growth medium: StemPro® hESC SFM (defined, serum- and feeder-free medium (SFM)) supplemented with Nicotinamide and Y27632 (Rho-associated, coiled-coil containing protein kinase (ROCK-1) inhibitor).

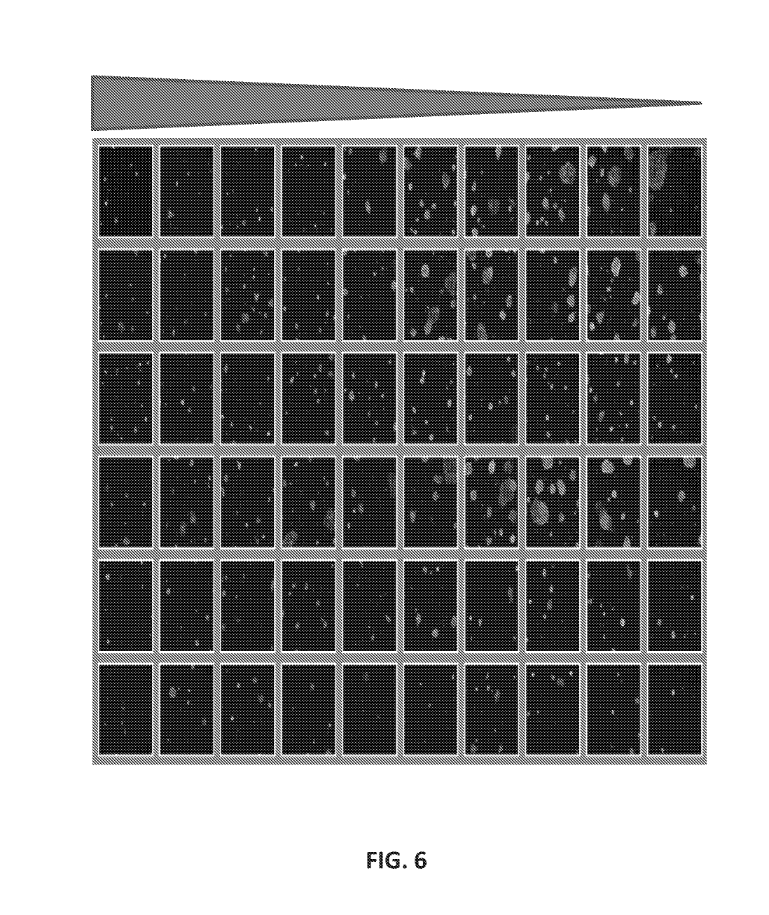

For ex vivo drug testing, the seeded tumor cells were exposed to drug or drugs combination for 72 hours. The drugs were prepared in DMSO and the final concentration of DMSO in media was 0.1%. The drug and drug combinations were tested in a ten-point serial dilution (see FIG. 6). Following exposure with drug, the epithelial tumor cells were labeled with 5-ethynyl-2′-deoxyuridine (Edu) to assess the tumor cell proliferation rates. The labeling lasted 24 hours in the presence of drug exposure. In the control group, epithelial tumor cells received no drug exposure with media change, but were similarly labeled with Edu. The labeled tumor cell were fixed and stained with Hoechst 33342 in blocking buffer containing 0.5% Triton X-100 and 3% BSA overnight at 4° C. The tumor cells were incubated with EpCAM antibody (1:4000) for 2 hours at room temperature and rinsed with PBST. Subsequently, the tumor cells were incubated with Alexa Fluor® 647 conjugated goat-anti-mouse secondary antibody for 30 minutes at room temperature and rinsed with PBST.

The incorporated Edu was detected by Click-iT reaction where fixed cells were incubated with a reaction mixture containing 1× Click-iT Edu reaction buffer, CuSO4, and azide-conjugated Alexa Fluor dye in the dark. The stained cells were washed with PBS times before distributed into a black wall plate for image acquisition and analysis.

For normal tissues, no tumor clusters were observed under the above partial enzymatic digestion. Single cells were collected and seeded in a feeder cell and Rock inhibitor Y27632 at 3000 cells per well. The normal cells were grown for 72 hours before labeled with 5-ethynyl-2′-deoxyuridine (Edu) to assess the tumor cell proliferation rates. The normal cells were stained with EpCAM antibody to identify epithelial cells.

For image acquisition and analysis, the stained tumor cells were imaged by a high-content screening (HCS) platform (Thermo Scientific Cellomics Array Scan XTi HCS reader). The 10× objective was used to collect images. Twenty-five fields were imaged for each well, and more than 3000 cells were captured for the analysis. From the images three fluorescent signals were obtained from the HCS reader. Blue fluorescent signals recorded nucleus signals stained with Hoechst 33342, green fluorescent signal detected the Edu incorporated in newly synthesized DNA, and red fluorescent signal detected the EpCAM positive epithelial cells population. The Edu positive subpopulation cells percentage was calculated as percent of the total epithelial cell counts.

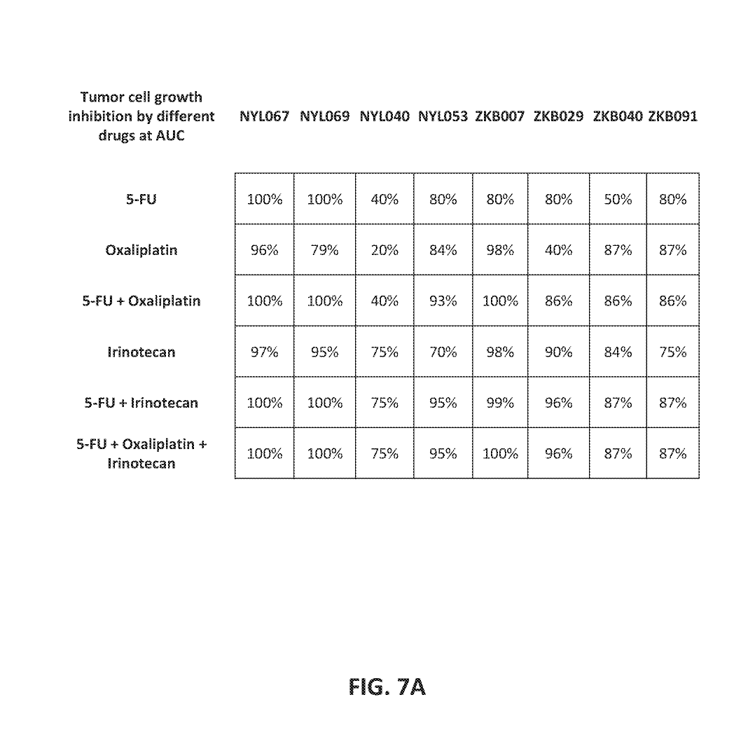

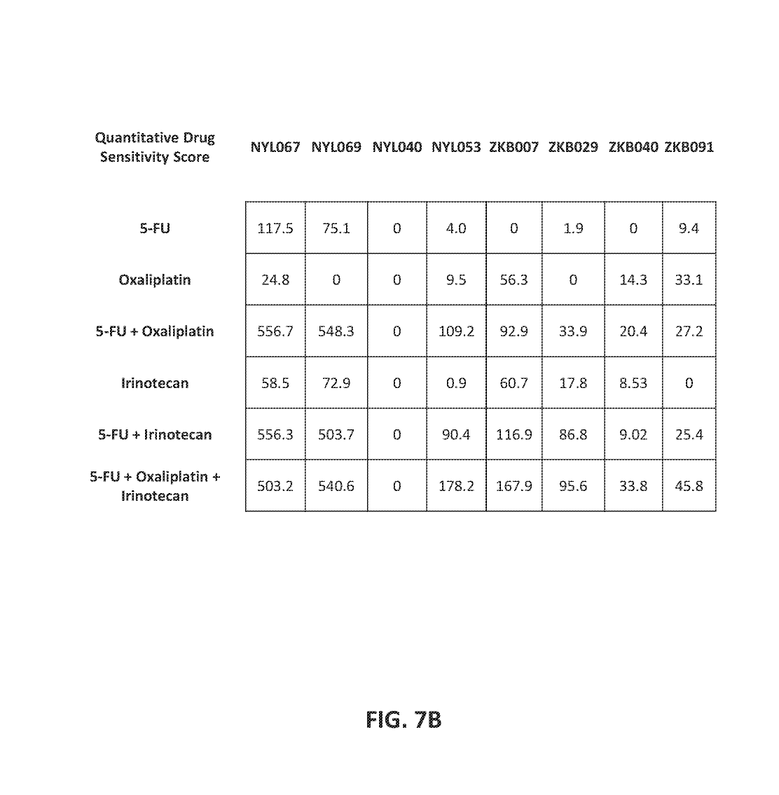

As shown in FIGS. 7A-7B, dose response data of Edu incorporated percentage for drugs and drugs combination were generated for eight different patients along with a quantitative drug sensitivity score (7B). Data can be represented by a logistic sigmoidal function with a maximum effect level (A max), the concentration at half-maximum activity of compound (EC50), a Hill coefficient representing the signorial transition, and a quantitative scoring approach to capture the multi-parametric dose-response relationship based upon activity area (DSS) to identify selective drug-response pattern.

A max (EdU)=MIc max/MIc

DSS(AUC)=∫MI(x)dx

The A max, EC₅₀, and DSS could be used to establish a predict model for the patient response to the tested drugs or drug combination.

1. A cell culture medium, comprising
(a) human ex vivo-derived cancer-tissue originated spheroids (CTOSs) which are dissociated into a substantially single cell suspension, wherein the CTOSs comprise human epithelial tumor cells,
(b) feeder cells; and
(c) a defined cell culture medium that comprises at least one Rho-associated, coiled-coil containing protein kinase (ROCK) inhibitor, optionally wherein the defined cell culture medium does not comprise or is substantially free of a Bone Morphogenetic Protein (BMP) inhibitor such as Noggin and a Wnt/β-catenin signaling agonist such as R-spondin-1; and optionally
(d) supplemental serum albumin (SA),
wherein the cell culture medium provides at least about 10% proliferation of the epithelial tumor cells within about 14 days following co-culture of the dissociated CTOSs with (b) and (c).

2. The cell culture medium of claim 1, wherein the human ex vivo-derived CTOSs are cultured from a tumor-containing sample removed from a human patient with cancer, optionally selected from a surgical sample, a biopsy sample, a pleural effusion sample, and an ascetic fluid sample.

3. The cell culture medium of claim 1 or 2, wherein the human patient has a cancer selected from colon cancer, lung cancer, gastric cancer, and breast cancer.

4. The cell culture medium of claim 2 or 3, wherein the epithelial tumor cells substantially retain the clonal diversity of the tumor-containing sample removed from the human patient within about 14 days following co-culture of the dissociated CTOSs with (b) and (c).

5. The cell culture medium of any one of claims 1-4, wherein the human epithelial tumor cells are selected from colon cancer cells, lung cancer cells, gastric cancer cell, and breast cancer cells.

6. The cell culture medium of any one of claims 1-5, which provides at least about 10, 20, 30, 40, 50, 60, 70, 80, 90, or 100% proliferation of the human epithelial tumor cells within about 14, 13, 12, 11, 10, 9, 8, 7, 6, 5, 4, 3, 2 or 1 days following co-culture of the dissociated CTOSs with (b) and (c).

7. The cell culture medium of any one of claims 1-6, which provides at least about 20-30% proliferation of the human epithelial tumor cells within about 7 days following co-culture of the dissociated CTOSs with (b) and (c).

8. The cell culture medium of any one of claims 1-7, which provides at least about 60% proliferation of the human epithelial tumor cells within about 7 days following co-culture of the dissociated CTOSs with (b) and (c).

9. The cell culture medium of any one of claims 1-8, which provides a ratio of about or at least about 5:1, 10:1, 20:1, 50:1, 100:1, 200:1, 500:1, or 1000:1 between the human epithelial tumor cells and human stromal cells within about 14 days following co-culture of the dissociated CTOSs with (b) and (c), wherein the human epithelial tumor cells are optionally characterized by cell surface expression of epithelial cell adhesion molecule (EpCAM) and/or CD133.

10. The cell culture medium of any one of claims 1-9, wherein the defined medium comprises serum, optionally fetal bovine serum (FBS).

11. The cell culture medium of claim 10, wherein the concentration of the serum ranges from about 1-5%, or is about 1%, 2%, 3%, 4%, or 5%.

12. The cell culture medium of any one of claims 1-11, wherein the ROCK inhibitor is selected from one or more of Y27632, HA1100 hydrochloride, HA1077, and GSK429286.

13. The cell culture medium of claim 12, wherein the concentration of Y27632 ranges from about 0.1-1000, 0.5-1000, 1-1000, 5-1000, 10-1000, 50-1000, 100-1000, 500-1000 μM, or from about 0.1-500, 0.5-500, 1-500, 5-500, 10-500, 50-500, 100-500 μM, or from about 0.1-100, 0.5-100, 1-100, 5-100, 10-100, 50-100 μM, or from about 0.1-50, 0.5-50, 1-50, 5-50, 10-50 μM, or from about 0.1-40, 0.5-40, 1-40, 5-40, 10-40 μM, or from about 0.1-30, 0.5-30, 1-30, 5-30, 10-30 μM, or from about 0.1-20, 0.5-20, 1-20, 5-20, 10-20 μM, or from about 0.1-10, 0.5-10, 1-10, 5-10 μM, or from about 0.1-5, 0.5-5, 1-5 μM, or from about 0.1-1 or 0.5-1 μM.

14. The cell culture medium of any one of claims 1-13, wherein the supplemental SA is selected from bovine serum albumin (BSA) and human serum albumin (HSA).

15. The cell culture medium of any one of claims 1-14, wherein the concentration of the supplemental SA is from about 0.5 mg/ml to about 20 mg/ml, or about, less than about, or no more than about 0.5, 0.6, 0.7, 0.8, 0.9, 1.0, 1.2, 1.4, 1.6, 1.8, 2.0, 2.2, 2.5, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, 10.0, 10.2, 10.4, 10.6, 10.8, 11.0, 11.2, 11.4, 11.6, 11.8, 12.0, 12.2, 12.4, 12.6, 12.8, 13.0, 13.2, 13.4, 13.6, 13.8, 14.0, 14.2, 14.4, 14.6, 14.8, 15.0, 15.2, 15.4, 15.6, 15.8, 16.0, 16.2, 16.4, 16.6, 16.8, 17.0, 17.2, 17.4, 17.6, 17.8, 18.0, 18.2, 18.4, 18.6, 18.8, 19.0, 19.2, 19.4, 19.6, 19.8, or 20 mg/ml.

16. The cell culture medium of any one of claims 1-15, wherein the feeder cells are non-proliferating fibroblasts, optionally human fibroblasts.

17. A tissue culture plate having a low cell binding surface, comprising a cell culture medium of any one of claims 1-16.

18. A method of culturing or expanding human epithelial tumor cells in an in vitro cell culture medium, comprising
(A) dissociating human ex vivo-derived cancer-tissue originated spheroids (CTOSs) which comprise the human epithelial tumor cells into a substantially single cell suspension; and
(B) co-culturing the dissociated CTOSs with feeder cells in a defined cell culture medium that comprises at least one Rho-associated, coiled-coil containing protein kinase (ROCK) inhibitor, optionally wherein the defined cell culture medium does not comprise or is substantially free of a Bone Morphogenetic Protein (BMP) inhibitor such as Noggin and a Wnt/γ-catenin signaling agonist such as R-spondin-1, and optionally wherein the cell culture medium comprises supplemental serum albumin (SA), wherein the method provides at least about 10% proliferation of the epithelial tumor cells in the cell culture medium within about 14 days following steps (A) and (B).

19. The method of claim 18, wherein the substantially dissociated CTOSs are co-cultured in a tissue culture plate having a low cell binding surface.

20. The method of claim 19, wherein the human ex vivo-derived CTOSs are cultured from a tumor sample removed from a human patient with cancer, optionally selected from a surgical sample, a biopsy sample, a pleural effusion sample, and an ascetic fluid sample.

21. The method of claim 19 or 20, wherein the human patient has a cancer selected from colon cancer, lung cancer, gastric cancer, and breast cancer.

22. The method of claim 18 or 19, wherein the epithelial tumor cells substantially retain the clonal diversity of the tumor sample removed from the human patient within about 14 days following steps (A) and (B).

23. The method of any one of claims 18-22, wherein the human epithelial tumor cells are selected from colon cancer cells, lung cancer cells, gastric cancer cell, and breast cancer cells.

24. The method of any one of claims 18-23, which provides at least about 10, 20, 30, 40, 50, 60, 70, 80, 90, or 100% proliferation of the human epithelial tumor cells in the cell culture medium within about 14, 13, 12, 11, 10, 9, 8, 7, 6, or 5 days following steps (A) and (B).

25. The method of any one of claims 18-24, which provides at least about 20-30% proliferation of the human epithelial tumor cells in the cell culture medium within about 7 days following steps (A) and (B).

26. The method of any one of claims 18-25, which provides at least about 60% proliferation of the human epithelial tumor cells in the cell culture medium within about 7 days following steps (A) and (B).

27. The method of any one of claims 18-26, which provides a ratio of at least about 5:1, 10:1, 20:1, 50:1, 100:1, 200:1, 500:1, or 1000:1 between the human epithelial tumor cells and human stromal cells in the cell culture medium within about 14 days following steps (A) and (B), wherein the human epithelial tumor cells are optionally characterized by cell surface expression of epithelial cell adhesion molecule (EpCAM) and/or CD133.

28. The method of any one of claims 18-27, wherein the defined cell culture medium comprises serum, optionally fetal bovine serum (FBS).

29. The method of claim 28, wherein the concentration of the serum ranges from about 1-5%, or is about 1%, 2%, 3%, 4%, or 5%.

30. The method of any one claims 18-29, wherein the ROCK inhibitor is selected from one or more of Y27632, HA1100 hydrochloride, HA1077, and GSK429286.

31. The method of claim 30, wherein the concentration of Y27632 ranges from about 0.1-1000, 0.5-1000, 1-1000, 5-1000, 10-1000, 50-1000, 100-1000, 500-1000 μM, or from about 0.1-500, 0.5-500, 1-500, 5-500, 10-500, 50-500, 100-500 μM, or from about 0.1-100, 0.5-100, 1-100, 5-100, 10-100, 50-100 μM, or from about 0.1-50, 0.5-50, 1-50, 5-50, 10-50 μM, or from about 0.1-40, 0.5-40, 1-40, 5-40, 10-40 μM, or from about 0.1-30, 0.5-30, 1-30, 5-30, 10-30 μM, or from about 0.1-20, 0.5-20, 1-20, 5-20, 10-20 μM, or from about 0.1-10, 0.5-10, 1-10, 5-10 μM, or from about 0.1-5, 0.5-5, 1-5 μM, or from about 0.1-1 or 0.5-1 μM.

32. The cell culture medium of any one of claims 18-31, wherein the supplemental SA is selected from bovine serum albumin (BSA) and human serum albumin (HSA).

33. The cell culture medium of any one of claims 18-32, wherein the concentration of the supplemental SA is from about 0.5 mg/ml to about 20 mg/ml, or about, less than about, or no more than about 0.5, 0.6, 0.7, 0.8, 0.9, 1.0, 1.2, 1.4, 1.6, 1.8, 2.0, 2.2, 2.5, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, 10.0, 10.2, 10.4, 10.6, 10.8, 11.0, 11.2, 11.4, 11.6, 11.8, 12.0, 12.2, 12.4, 12.6, 12.8, 13.0, 13.2, 13.4, 13.6, 13.8, 14.0, 14.2, 14.4, 14.6, 14.8, 15.0, 15.2, 15.4, 15.6, 15.8, 16.0, 16.2, 16.4, 16.6, 16.8, 17.0, 17.2, 17.4, 17.6, 17.8, 18.0, 18.2, 18.4, 18.6, 18.8, 19.0, 19.2, 19.4, 19.6, 19.8, or 20 mg/ml.

34. The method of any one of claims 18-33, wherein the feeder cells are non-proliferating fibroblasts, optionally human fibroblasts.

35. The method of any one of claims 18-34, wherein the human ex vivo-derived CTOSs are cultured from a tumor-containing sample removed from a human patient with cancer, which optionally comprises
mincing and incubating the tumor-containing sample in a tissue digestion medium, and
culturing the tumor-containing sample in a defined, serum-free, and feeder-free CTOS growth medium that comprises nicotinamide, Wnt3A, a Bone Morphogenetic Protein (BMP) inhibitor, a Wnt/β-catenin signaling agonist, and a ROCK inhibitor, for a time sufficient to form the CTOSs.

36. The method of claim 35, wherein the tissue digestion medium comprises a protease selected from one or more of collagenase I, collagenase II, a neutral non-clostridial protease, and any combination thereof.

37. The method of claim 35 or 36, wherein the concentration of nicotinamide ranges from about 0.1-1000, 0.5-1000, 1-1000, 5-1000, 10-1000, 50-1000, 100-1000, 500-1000 mM, or from about 0.1-500, 0.5-500, 1-500, 5-500, 10-500, 50-500, 100-500 mM, or from about 0.1-100, 0.5-100, 1-100, 5-100, 10-100, 50-100 mM, or from about 0.1-50, 0.5-50, 1-50, 5-50, 10-50 mM, or from about 0.1-40, 0.5-40, 1-40, 5-40, 10-40 mM, or from about 0.1-30, 0.5-30, 1-30, 5-30, 10-30 mM, or from about 0.1-20, 0.5-20, 1-20, 5-20, 10-20 mM, or from about 0.1-10, 0.5-10, 1-10, 5-10 mM, or from about 0.1-5, 0.5-5, 1-5 mM, or from about 0.1-1 or 0.5-1 mM.

38. The method of any one of claims 35-37, wherein the concentration of Wnt3A ranges from about 0.1-10,000 or 1-1000 ng/ml, or from about 0.1-1000, 1-1000, 10-1000, 20-1000, 30-1000, 40-1000, 50-1000, 60-1000, 70-1000, 80-1000, 90-1000, 100-1000, 200-1000, 300-1000, 400-1000, 500-1000, 600-1000, 700-1000, 800-1000, 900-1000 ng/ml, or from about 0.1-500, 1-500, 10-500, 20-500, 30-500, 40-500, 50-500, 60-500, 70-500, 80-500, 90-500, 100-500, 200-500, 300-500, 400-500 ng/ml, or from about 0.1-400, 1-400, 10-400, 20-400, 30-400, 50-400, 40-400, 60-400, 70-400, 80-400, 90-400, 100-400, 200-400, 300-400 ng/ml, or from about 0.1-300, 1-300, 10-300, 20-300, 30-300, 40-300, 50-300, 60-300, 70-300, 80-300, 90-300, 100-300, 200-300 ng/ml, or from about 0.1-200, 1-200, 10-200, 20-200, 30-200, 40-200, 50-200, 60-200, 70-200, 80-200, 90-200, 100-200 ng/ml, or from about 0.1-150, 1-150, 10-150, 20-150, 30-150, 40-150, 50-150, 60-150, 70-150, 80-150, 90-150, 100-150 ng/ml, or from about 0.1-120, 1-120, 10-120, 20-120, 30-120, 40-120, 50-120, 60-120, 70-120, 80-120, 90-120, 100-120 ng/ml, or from about 0.1-100, 1-100, 10-100, 20-100, 30-100, 40-100, 50-100, 60-100, 70-100, 80-100, 90-100 ng/ml.

39. The method of any one of claims 35-38, wherein the BMP inhibitor is noggin, wherein the concentration of noggin optionally ranges from about 0.1-10,000 or 1-1000 ng/ml, or from about 0.1-1000, 1-1000, 10-1000, 20-1000, 30-1000, 40-1000, 50-1000, 60-1000, 70-1000, 80-1000, 90-1000, 100-1000, 200-1000, 300-1000, 400-1000, 500-1000, 600-1000, 700-1000, 800-1000, 900-1000 ng/ml, or from about 0.1-500, 1-500, 10-500, 20-500, 30-500, 40-500, 50-500, 60-500, 70-500, 80-500, 90-500, 100-500, 200-500, 300-500, 400-500 ng/ml, or from about 0.1-400, 1-400, 10-400, 20-400, 30-400, 50-400, 40-400, 60-400, 70-400, 80-400, 90-400, 100-400, 200-400, 300-400 ng/ml, or from about 0.1-300, 1-300, 10-300, 20-300, 30-300, 40-300, 50-300, 60-300, 70-300, 80-300, 90-300, 100-300, 200-300 ng/ml, or from about 0.1-200, 1-200, 10-200, 20-200, 30-200, 40-200, 50-200, 60-200, 70-200, 80-200, 90-200, 100-200 ng/ml, or from about 0.1-150, 1-150, 10-150, 20-150, 30-150, 40-150, 50-150, 60-150, 70-150, 80-150, 90-150, 100-150 ng/ml, or from about 0.1-120, 1-120, 10-120, 20-120, 30-120, 40-120, 50-120, 60-120, 70-120, 80-120, 90-120, 100-120 ng/ml, or from about 0.1-100, 1-100, 10-100, 20-100, 30-100, 40-100, 50-100, 60-100, 70-100, 80-100, 90-100 ng/ml.

40. The method of any one of claims 35-39, wherein the Wnt/β-catenin signaling agonist is R-spondin-1, wherein the concentration of R-spondin-1 optionally ranges from about 0.1-10,000 or 1-1000 or 100-1000 ng/ml, or from about 0.1-10,000, 1-10,000, 10-10,000, 20-10,000, 30-10,000, 40-10,000, 50-10,000, 60-10,000, 70-10,000, 80-10,000, 90-10,000, 100-10,000, 200-10,000, 300-10,000, 400-10,000, 500-10,000, 1000-10,000, 5000-10,000 ng/ml, or from about 0.1-5000, 1-5000, 10-5000, 20-5000, 30-10,000, 40-5000, 50-5000, 60-5000, 70-5000, 80-5000, 90-5000, 100-5000, 200-5000, 300-5000, 400-5000, 500-5000, 1000-5000 ng/ml, or from about 0.1-1000, 1-1000, 10-1000, 20-1000, 30-1000, 40-1000, 50-1000, 60-1000, 70-1000, 80-1000, 90-1000, 100-1000, 200-1000, 300-1000, 400-1000, 500-1000, 600-1000, 700-1000, 800-1000, 900-1000 ng/ml, or from about 0.1-800, 1-800, 10-800, 20-800, 30-800, 40-800, 50-800, 60-800, 70-800, 80-800, 90-800, 100-800, 200-800, 300-800, 400-800, 500-800, 600-800, 700-800 ng/ml, or from about 0.1-700, 1-700, 10-700, 20-700, 30-700, 40-700, 50-700, 60-700, 70-700, 80-700, 90-700, 100-700, 200-700, 300-700, 400-700, 500-700, 600-700 ng/ml, or from about 0.1-600, 1-600, 10-600, 20-600, 30-600, 40-600, 50-600, 60-600, 70-600, 80-600, 90-600, 100-600, 200-600, 300-600, 400-600, 500-600 ng/ml, or from about 0.1-500, 1-500, 10-500, 20-500, 30-500, 40-500, 50-500, 60-500, 70-500, 80-500, 90-500, 100-500, 200-500, 300-500, 400-500 ng/ml.

41. The method of any one of claims 35-40, wherein the ROCK inhibitor is Y27632, wherein the concentration of Y27632 optionally ranges from about 0.1-1000, 0.5-1000, 1-1000, 5-1000, 10-1000, 50-1000, 100-1000, 500-1000 μM, or from about 0.1-500, 0.5-500, 1-500, 5-500, 10-500, 50-500, 100-500 μM, or from about 0.1-100, 0.5-100, 1-100, 5-100, 10-100, 50-100 μM, or from about 0.1-50, 0.5-50, 1-50, 5-50, 10-50 μM, or from about 0.1-40, 0.5-40, 1-40, 5-40, 10-40 μM, or from about 0.1-30, 0.5-30, 1-30, 5-30, 10-30 μM, or from about 0.1-20, 0.5-20, 1-20, 5-20, 10-20 μM, or from about 0.1-10, 0.5-10, 1-10, 5-10 μM, or from about 0.1-5, 0.5-5, 1-5 μM, or from about 0.1-1 or 0.5-1 μM.

42. A method of testing responsiveness of a human patient to a therapeutic agent, comprising
administering the therapeutic agent to a cell culture medium of any one of claims 1-15, or to a cell culture medium prepared by a method of any one of claims 17-41; and
measuring epithelial tumor cell proliferation and/or epithelial tumor cell apoptosis,
wherein a decrease in epithelial tumor cell proliferation and/or an induction in epithelial tumor cell apoptosis is indicative of responsiveness of the human patient to the therapeutic agent, and wherein a lack of decrease in epithelial tumor cell proliferation and/or induction of epithelial tumor cell apoptosis is indicative of resistance of the human patient to the therapeutic agent.

43. The method of claim 42, comprising administering the therapeutic agent on the same day as co-culturing the dissociated CTOSs.

44. The method of claim 42, comprising administering the therapeutic agent at least one day following co-culture of the dissociated CTOSs.

45. The method of claim 44, comprising administering the therapeutic agent about or within about 1, 2, 3, 4, 5, 6, or 7 days following co-culture of the dissociated CTOSs.

46. The method of any one of claims 43-45, comprising measuring epithelial tumor cell proliferation and/or epithelial tumor cell apoptosis within about 14 days of administering the therapeutic agent.

47. The method of claim 46, comprising measuring epithelial tumor cell proliferation and/or epithelial tumor cell apoptosis within about 14, 13, 12, 11, 10, 9, 8, 7, 6, or 5 days of administering the therapeutic agent.

48. The method of claim 47, comprising measuring epithelial tumor cell proliferation and/or epithelial tumor cell apoptosis within about 7, 6, or 5 days of administering the therapeutic agent.

49. The method of any one of claims 42-48, wherein the step of measuring epithelial tumor cell proliferation comprises measuring a cellular proliferation marker.

50. The method of claim 49, wherein the cellular proliferation marker is selected from one or more of ³H-thymidine, bromodeoxyuridine (BrdU), 5-ethynyl-2′-deoxyuridine (Edu), Ki-67, and proliferating cell nuclear antigen (PCNA).

51. The method of any one of claims 42-50, wherein the step of measuring epithelial tumor cell apoptosis comprises measuring a cellular apoptosis marker.

52. The method of claim 51, wherein the cellular apoptosis marker is selected from one or more of fluorochrome-labeled inhibitors of Caspases (FLICA), caspase activation, poly ADP ribose polymerase (PARP) cleavage, DRAQ5, DRAQ7, and terminal deoxynucleotidyl transferase (TdT) dUTP nick-end labeling (TUNEL) assay.

53. A sample preparation kit, comprising any combination of:
a defined, serum-free, and feeder-free cancer-tissue originated spheroid (CTOSs) growth medium;
a tissue digestion medium;
a tissue culture plate having a low cell binding surface;
a defined cell culture medium, which optionally comprises supplemental serum albumin (SA), and optionally wherein the defined cell culture medium does not comprises or is substantially free of a Bone Morphogenetic Protein (BMP) inhibitor such as Noggin and a Wnt/β-catenin signaling agonist such as R-spondin-1;
frozen feeder cells;
a Rho-associated, coiled-coil containing protein kinase (ROCK) inhibitor; optionally
a cellular proliferation marker and/or a cellular apoptosis marker.

54. The sample preparation kit of claim 53, wherein the CTOS growth medium comprises nicotinamide, Wnt3A, a Bone Morphogenetic Protein (BMP) inhibitor, a Wnt/β-catenin signaling agonist, and/or a ROCK inhibitor.

55. The sample preparation kit of claim 53 or 54, wherein the tissue digestion medium comprises a protease selected from one or more of collagenase I, collagenase II, a neutral non-clostridial protease, and any combination thereof.

56. The sample preparation kit of any one of claims 53-55, wherein the defined culture medium comprises serum, optionally fetal bovine serum (FBS), at a concentration that ranges from about 1-5%, or that is about 1%, 2%, 3%, 4%, or 5%.

57. The sample preparation kit of any one of claims 53-56, wherein the frozen feeder cells are non-proliferating fibroblasts, optionally human fibroblasts.

58. The sample preparation kit of any one of claims 53-57, wherein the ROCK inhibitor is selected from one or more of Y27632, HA1100 hydrochloride, HA1077, and GSK429286.

59. The sample preparation kit of any one of claims 53-58, wherein the cellular proliferation marker is selected from one or more of ³H-thymidine, bromodeoxyuridine (BrdU), 5-ethynyl-2′-deoxyuridine (Edu), Ki-67, and proliferating cell nuclear antigen (PCNA).

60. The sample preparation kit of any one of claims 53-59, wherein the cellular apoptosis marker is selected from one or more of fluorochrome-labeled inhibitors of Caspases (FLICA), caspase activation, poly ADP ribose polymerase (PARP) cleavage, DRAQ5, DRAQ7, and terminal deoxynucleotidyl transferase (TdT) dUTP nick-end labeling (TUNEL) assay.
